(19) United States
(12) Patent Application Publication
(10) Pub. No.: US 2005/0272917 A1
(43) Pub. Date: Dec. 8, 2005

(54) Methods for immunoglobulin purification

(75) Inventors: Jin-An Jiao, Sioux Falls, SD (US); Scott Fulton, Middleton, WI (US)

Correspondence Address: CLARK & ELBING LLP, 101 FEDERAL STREET, BOSTON, MA, 02110, US

(73) Assignees: Hematech, LLC, Westport, CT (US); Kirin Beer Kabushiki Kaisha, Chuo-Ku (JP)

(21) Appl. No.: 11/129,095

(22) Filed: May 13, 2005

Related U.S. Application Data
(60) Provisional application No. 60/571,146, filed on May 14, 2004.

(51) Int. Cl. C07K 16/18

(52) U.S. Cl. 530/388.1

(57) ABSTRACT

Disclosed herein are methods for purifying immunoglobulin G (IgG). The methods feature the use of particular buffers and reagents to isolate and purify human IgG or to remove host contaminating proteins, non-human or chimeric IgG, IgG dimers, IgG aggregates, bovine serum albumin, transmissible spongiform encephalopathy, DNA, viral DNA, or viral particles from a feedstock. IgG purified by the methods described herein can be used for research, diagnostic, or therapeutic purposes.

### CROSS-REFERENCE TO RELATED APPLICATIONS

This application claims the benefit of the filing date of U.S. provisional application 60/571,146, filed May 14, 2004, herein incorporated by reference.

### BACKGROUND OF THE INVENTION

The invention generally relates to methods for purifying immunoglobulins.

Immunoglobulin (Ig) is extremely important for use in diagnostic and therapeutic fields. For example, immunoglobulin G (IgG) preparations isolated from human plasma or hyperimmune plasma or sera have been used to treat diseases such as inherited and acquired immune-deficiency diseases and infectious diseases.

Generally, immunoglobulin is obtained from animal sera or from cultivation of suitable cell lines. Previously used methods for IgG purification from plasma or sera using Cohn ethanol fractionation followed by ion exchange chromatography or caprylic acid (CA) precipitation have been described (see for example McKinney et al. J. Immunol. Methods 96:271-278, 1987; U.S. Pat. Nos. 4,164,495; 4,177,188; RE 31,268; U.S. Pat. Nos. 4,939,176; and 5,164,487). These previously described methods generally require the use of low concentrations of CA (0.4% to 2.5%) and some also required the use of additional precipitation steps such as ammonium sulfate precipitation. In addition, the feedstock was often very dilute, resulting in large feedstock volumes. These conventional methods of production and purification can suffer from limitations such as contamination of the purified product, insufficient yield, and a high cost of producing antibodies on a large scale.

Therefore, there exists a need for improved methods for purification of IgGs from animal fluids such as plasma or serum, with a high purity and high yield, generally free of IgG dimers and aggregates, as well as viral and host protein contaminants.

### SUMMARY OF THE INVENTION

We have discovered improved methods for purifying IgG from wild-type or transgenic animal fluids such as plasma, serum, ascites, and milk, or cell culture supernatants containing polyclonal or monoclonal antibodies.

In a first aspect, the invention features a method for purifying IgG from a feedstock. The method includes several steps. First, the pH of the feedstock is adjusted to be in a range of about 4.0 to 5.5 (e.g., pH 4.0, 4.2, 4.4, 4.5, 4.6, 4.8, or 5.0). The pH-adjusted feedstock is then contacted with a mono or polyalkanoic acid having between 4 and 12 carbon atoms, such as any alkanoic acid having between 4 and 12 carbon atoms, and preferably from 6 to 9 carbon atoms. Desirably, the alkanoic acid is CA. Although unbranched alkanoic acids are preferable, branched alkanoic acids can also be used. When using higher alkanoic acids, such as those having from 9 to 12 carbon atoms, it may be advantageous to incorporate additionally one or more carboxyl groups to improve water solubility. The same effect is achieved by using alkanoic acids having substituents containing, for example, one or more hydroxyl groups or amino groups. In this method, the acid (e.g., CA) is added in an amount and for a time sufficient to form a precipitate, where the supernatant solution contains the IgG. The alkanoic acid concentration can be calculated as a percent of the total volume and can be determined empirically for each feedstock. Desirably, when the alkanoic acid is CA, the CA concentration in the feedstock solution is at least 3%, 4%, 5%, 6%, 7%, 8%, 9%, 10%, or more. The alkanoic acid concentration can also be calculated relative to the amount of total protein in the feedstock. For example, total protein concentration of the feedstock can be determined using standard protein assay methods (e.g., the BCA assay kit from Pierce Biotechnology Inc. or the Bradford assay kit from Bio-Rad) and alkanoic acid is added in an amount such that the ratio of alkanoic acid/total protein is about 0.75 to 2.25, preferably about 1 to 2.25.

After the addition of alkanoic acid, the supernatant containing the IgG is separated from the precipitate (e.g., by centrifugation or filtration). The pH of the supernatant is then adjusted to be within a neutral pH range or to a pH that is suitable for the chromatography resin used in the subsequent step. The supernatant is then contacted with at least one chromatography reagent with an affinity for IgG under conditions that allow binding to the reagent of the IgG in the supernatant solution. Suitable resins include any resin with an affinity for IgG (e.g., resins with Protein A, Protein G, Protein L, 4-Mercapto-Ethyl-Pyridine, or anti-human IgG antibodies (e.g., horse anti-human IgG or llama anti-human IgG) as the ligand). The ligand can be a naturally-occurring protein or a recombinant or synthetically produced ligand. Exemplary chromatography resins are: Protein A-Sepharose™, Protein A-agarose, Protein A-agarose CL-4B, Protein G-Sepharose™, Protein G-agarose, Protein G-agarose CL-4B, Protein L-agarose, Protein A/G agarose, KAPTIV™ immunoaffinity matrices (e.g., KAPTIV-GY™, KAPTIV-AE™, KAPTIV-M™, all from Tecnogen, Inc.), Cellthru BigBead™ (Sterogene), Protein A Ultraflow™ (Sterogene), Protein A Cellthru™ 300 (Sterogene), QuickMab (Sterogene), QuickProtein A™ (Sterogene), Thruput™ or Thruput Plus (Sterogene), PROSEP-A and PROSEP-G (Millipore), MEP Hypercel™ (Ciphergen), MBI Hypercel™ (Ciphergen), CM Hyperz™ (Ciphergen), and NHS-activated Sepharose™ 4 Fast Flow. The ligand may be also immobilized on a membrane support or cartridge such as the Mustang™ membrane from Pall Life Sciences.

The IgG is eluted from the chromatography resin using an eluent having a pH that is optimal for the resin used. In one example an eluent with an acidic pH of about 3.0 to 5.0 (e.g., pH 3.0, 3.5, 4.0, 4.2, 4.4, 4.5, 4.6, 4.8, or 5) is used. In another example, an eluent with a basic pH of about 8.0-11.0 is used. Optionally, the pH of the solution after elution (i.e., the eluate) is adjusted to a neutral pH.

The feedstock may be, for example, plasma, serum, ascites, or milk taken from a wild-type or transgenic mammal (e.g., ungulate, mouse, horse, pig, rat, and rabbit). Preferred ungulates are ovine, bovine, porcine, and caprine. In one embodiment, the mammal is a transgenic bovine that produces human IgGs. The feedstock can also be a cell culture supernatant containing polyclonal or monoclonal antibodies.

Preferably, this method yields a preparation of IgG that is at least 80%, 85%, 90%, 95%, or 99% or more pure. Desirably, the preparation of IgG has less than 5%, 4%, 3%, 2%, 1% or 0.5% of non-human IgG, less than 45%, 40%, 35%, 30%, 25% or, 20% chimeric IgG, less than 100, 50, or 10 ppm bovine serum albumin, less than 500, 250, or 100 ppm host contaminating proteins, or less than 5, 4, 3, 2, or 1 ppm DNA, or undetectable levels of transmissible spongiform encephalopathy, viral DNA, or viral particles using standard methods of detection known in the art, such as Western blot analysis, infectivity assays, or PCR analysis.

In a second aspect, the invention features a method for purifying IgG from a feedstock. This method includes several steps. First, the pH of the feedstock is adjusted to be in a range of about 4.0 to 5.5 (e.g., pH 4.0, 4.2, 4.4, 4.5, 4.6, 4.8, or 5.0). The pH-adjusted feedstock is then contacted with a mono or polyalkanoic acid having between 4 and 12 carbon atoms, such as any alkanoic acid having between 4 and 12 carbon atoms, and preferably from 6 to 9 carbon atoms as described above. Desirably, the alkanoic acid is CA. Desirably, when the alkanoic acid is CA, the CA concentration in the feedstock solution is at least 3%, 4%, 5%, 6%, 7%, 8%, 9%, 10%, or more. The alkanoic acid concentration can also be calculated relative to the amount of total protein in the feedstock. For example, total protein concentration of the feedstock can be determined using standard protein assay methods (e.g., the BCA assay kit from Pierce Biotechnology Inc. or the Bradford assay kit from Bio-Rad) and alkanoic acid is added in an amount such that the ratio of alkanoic acid/total protein is about 0.75 to 2.25, preferably about 1 to 2.25.

After the addition of alkanoic acid, the supernatant containing the IgG is separated from the precipitate (e.g., by centrifugation or filtration). The supernatant is then dialyzed against a buffer having a pH of about 4.5 to about 6.0, preferably about 5.0. Desirably, the buffer contains MOPSO and β-alanine. The IgG is then separated from the supernatant using membrane-mediated electrophoresis (e.g., the Gradiflow™ system) and the purified IgG is collected.

The feedstock may be, for example, plasma, serum, ascites, or milk taken from a wild-type or transgenic mammal (e.g., ungulate, mouse, horse, pig, rat, and rabbit). Preferred ungulates are ovine, bovine, porcine, and caprine. In one embodiment, the mammal is a transgenic bovine that produces human IgGs. The feedstock can also be the supernatant from a cell culture supernatant containing polyclonal or monoclonal antibodies.

Preferably, this method yields a preparation of IgG that is at least 80%, 85%, 90%, 95%, or 99% or more pure. Desirably the preparation of IgG has less than 5%, 4%, or 3% of IgG aggregates, less than 100, 50, or 10 ppm bovine serum albumin, less than 500, 250, or 100 ppm host contaminating proteins, or less than 5, 4, 3, 2, or 1 ppm DNA, or undetectable levels of transmissible spongiform encephalopathy, viral DNA, or viral particles using standard detection methods known in the art, such as Western blot analysis, infectivity assays or PCR analysis. This method can be used alone or as a final polishing step for further purification of IgG after any other method for IgG purification known in the art or described herein.

In a third aspect, the invention features a method for purifying human IgG from a feedstock containing both human IgG and non-human IgG, preferably obtained from a non-human transgenic mammal that expresses human IgG. This method includes several steps. First, the feedstock is contacted with at least one affinity chromatography resin that has Protein A as a ligand under conditions that allow binding of the human IgG to the resin. Desirably, the Protein A is a naturally occurring or a recombinant form of Protein A. The chromatography resin is then washed with a series of wash buffers having increasing acidity (e.g. pH 7.0, 6.5, 6.0, 5.8, 5.5, 5.2, 5.0, 4.8, 4.6, 4.5, 4.4, or 4.0) such that the washing causes the dissociation of non-human IgG from the resin but does not substantially dissociate human IgG. The resin can be washed at least one time, preferably at least two times, and most preferably at least three times with wash buffers, where the first wash buffer has a pH of about 5.0 to 6.0, preferably about 5.2, and each subsequent wash buffer has a pH that is more acidic than the previous wash buffer. In preferred embodiments, the wash buffers will not dissociate more than 20%, 10%, or 5% of the human IgG from the resin.

After washing the resin, the human IgG is eluted from the chromatography resin using an eluent having an acidic pH of about 2.5 to 3.5 (e.g., pH 2.5, 3.0, 3.5) and being more acidic than any of the wash buffers. The eluate contains the purified human IgG with a preferred purity of at least 80%, 85%, 90%, or 95% or more. Desirably the preparation of IgG has less than 5%, 4%, 3%, 2%, 1% or 0.5% of non-human IgG, less than 45%, 40%, 35%, 30%, 25% or 20% chimeric IgG, less than 100, 50, or 10 ppm bovine serum albumin, less than 500, 250, or 100 ppm host contaminating proteins, or less than 5, 4, 3, 2, or 1 ppm DNA, or undetectable levels of transmissible spongiform encephalopathy, viral DNA, or viral particles using standard methods of detection known in the art, such as Western blot analysis, infectivity assays or PCR analysis. The pH of the eluate is optionally adjusted to a neutral pH.

The feedstock used for the third aspect may be, for example, plasma, serum, ascites, or milk taken from a transgenic animal that expresses human IgG (e.g., ungulate, mouse, horse, pig, rat, and rabbit). Preferred ungulates are ovine, bovine, porcine, and caprine. In one embodiment, the mammal is a transgenic bovine that produces human IgGs. The feedstock may also be any feedstock that contains human IgG and has been previously purified according to the methods described above or any portion thereof.

In a fourth aspect, the invention features a method for purifying IgG monomers from a feedstock that includes IgG monomers and may also include IgG dimers or aggregates, or both. The first step of this method is to contact the feedstock with at least one chromatography resin that includes an antibody-selective ligand, under conditions that allow binding of at least some of the IgG monomer to the resin without substantially binding IgG dimer or aggregates, if present. The antibody-selective ligand includes a ligand that has a mercapto group and an aromatic pyridine ring (e.g., 4-Mercapto-Ethyl-Pyridine), and can also include a cellulose support (e.g., MEP HyperCel™ available from Ciphergen, catalog numbers 12035-069, 12035-010, 12035-028, 12035-036, 12035-040 and 12035-044). The resin is then washed with at least one wash buffer having a neutral or acidic pH, preferably ranging from about 5.5 to 9.0 (e.g., pH 6.0, 6.5, 7.0, 7.5, 8.0, or 8.5) and then the IgG monomer is eluted from the chromatography resin using an eluent having an acidic pH of about 3.0 to 5.0 (e.g., pH 3.0, 3.5, 4.0, 4.2, 4.4, 4.5, 4.6, 4.8, or 5.0).

The purified IgG monomer is in the eluent and desirably is at least 80%, 85%, 90%, 95% or more pure or at least 80%, 85%, 90%, 95% free of IgG dimers or aggregates. Desirably, the preparation of IgG has less than 100, 50, or 10 ppm bovine serum albumin, less than 500, 250, or 100 ppm host contaminating proteins, or less than 5, 4, 3, 2, or 1 ppm DNA, or undetectable levels of transmissible spongiform encephalopathy, viral DNA, or viral particles using standard methods of detection known in the art, such as Western blot analysis, infectivity assays, or PCR analysis.

The feedstock used for the fourth aspect may be, for example, plasma, serum, ascites, or milk taken from a wild-type or transgenic animal that expresses IgG monomers (e.g., ungulate, mouse, horse, pig, rat, and rabbit). Preferred ungulates are ovine, bovine, porcine, and caprine. In one embodiment, the mammal is a transgenic bovine that produces human IgGs or any feedstock previously purified by the methods described herein or any portion thereof (e.g., CA precipitation). The feedstock can also be the supernatant from a cell culture supernatant containing polyclonal or monoclonal antibodies. The feedstock can also be any feedstock in which IgG dimers, aggregates or both are generated during production and purification processes. Dimer or aggregate formation can be caused by changes in temperature (e.g., Pasteurization of serum of IgG samples), pH, exposure to certain chemicals such as ethanol during plasma fractionation step (e.g., Cohn fractions) and physical conditions such as gamma irradiation of plasma or serum during sterilization step. Dimer or aggregate formation can be caused by heating the feedstock to at least 50° C., preferably at least 55° C., and more preferably at least 60° C. prior to contacting the chromatography resin. In preferred embodiments, the feedstock is heated to 60° C. for at least thirty minutes, preferably one hour, most preferably two or more hours, and then cooled prior to performing step (a) of the method.

In a fifth aspect, the invention features a method for purifying human IgG from a feedstock that is taken from a transgenic non-human host that expresses human IgG (e.g., plasma, ascites, serum, or milk) or a feedstock taken from a transgenic ungulate and treated with CA as described for the methods above. Preferred ungulates are ovine, bovine, porcine, and caprine. In one embodiment, the mammal is a transgenic bovine that produces human IgGs. This method includes several steps. The first step of this method involves contacting the feedstock with at least one chromatography resin having an affinity for human IgG under conditions that allow the binding of the human IgG to the resin. The resin is then washed with at least one buffer that allows for the dissociation of non-human IgG from the resin but does not significantly dissociate human IgG (e.g., a buffer having an acidic pH of about 7.0, 6.5, 6.0, 5.8, 5.5, 5.2, 5.0, 4.8, 4.6, 4.5, 4.4, or 4.0). The resin can be washed at least one time, preferably at least two times, and most preferably at least three times with wash buffers, where the first wash buffer has a pH of about 5.0 to 6.0, preferably about 5.2. In preferred embodiments, the wash buffers will decrease in pH with each wash and will not dissociate more than 20%, 10%, or 5% of the human IgG from the resin. The human IgG is then eluted from the resin using an eluent having an acidic pH of about 2.5 to 3.5 (e.g., pH 2.5, 3.0, 3.5), and then optionally adjusted to a neutral pH. The eluate is contacted with at least one affinity chromatography resin comprising anti-host IgG as a ligand under conditions that allow binding of at least some of the non-human IgG to the resin, and the flow-through, which contains the purified human IgG, is then collected. The human IgG is preferably at least 80%, 85%, 90%, 95% or more pure or at least 80%, 85%, 90%, 95% or more free of non-human IgG, chimeric IgG, or both. The non-human or chimeric IgG can be optionally removed from the resin using an eluent.

Alternatively, the purification steps of the fifth aspect can be reversed so that the first step of the method involves contacting the feedstock with at least one affinity chromatography resin comprising anti-host IgG as a ligand under conditions that allow binding of the non-human IgG to the resin, and the flow-through, which contains the purified human IgG, is then collected. The flow-through is then contacted with at least one chromatography resin having an affinity for human IgG under conditions that allow the binding of the human IgG to the resin. The resin is then washed with at least one buffer that allows for the dissociation of non-human IgG from the resin but does not significantly dissociate human IgG (e.g., a buffer having an acidic pH of about 7.0, 6.5, 6.0, 5.8, 5.5, 5.2, 5.0, 4.8, 4.6, 4.5, 4.4, or 4.0). The resin can be washed at least one time, preferably at least two times, and most preferably at least three times with wash buffers having a pH of about 4.0 to 6.0, preferably starting with a buffer having a pH of about 5.2. In preferred embodiments, the wash buffers will decrease in pH with each wash and will not dissociate more than 20%, 10%, or 5% of the human IgG from the resin. The human IgG is then eluted from the resin using an eluent having an acidic pH of about 2.5 to 3.5 (e.g., pH 2.5, 3.0, 3.5), and then optionally adjusted to a neutral pH.

In desirable embodiments of each of these alternatives, the anti-host IgG ligand is, for example, a horse anti-bovine IgG or a ligand specific for the non-human host IgG heavy chain or light chain. Desirably, the ligand is a VHH ligand, which can, if desired, be prepared by the methods described in U.S. Patent Application Publication No. 20030078402, and U.S. Pat. Nos. 6,399,763 and 6,670,453. The chromatography resin can be any of the resins described herein. Suitable resins with an affinity for human IgG include resins with Protein A, Protein G, Protein L, 4-Mercapto-Ethyl-Pyridine, or anti-human IgG antibodies (e.g., horse anti-human IgG or llama anti-human IgG) as the ligand). The ligand can be a naturally-occurring protein or a recombinant or synthetically produced ligand.

Preferably, for either alternative this method yields a preparation of IgG that is at least 80%, 85%, 90%, 95%, or 99% or more pure. Desirably, the preparation of IgG has less than 5%, 4%, 3%, 2%, 1% or 0.5% of non-human IgG, less than 45%, 40%, 35%, 30%, 25% or 20% chimeric IgG, less than 100, 50, or 10 ppm bovine serum albumin, less than 500, 250, or 100 ppm host contaminating proteins, or less than 5, 4, 3, 2, or 1 ppm DNA, or undetectable levels of transmissible spongiform encephalopathy, viral DNA, or viral particles using detection methods known in the art, such as Western blot analysis, infectivity assays, or PCR analysis.

In a sixth aspect, the invention features a method for purifying human IgG from a feedstock that is taken from a transgenic non-human host that expresses human IgG (e.g., plasma, ascites, serum, or milk taken from a transgenic ungulate and treated with CA as described for the methods above). Preferred ungulates are ovine, bovine, porcine, and caprine. In one embodiment, the mammal is a transgenic bovine that produces human IgGs. This method includes several steps. The first step of this method involves contacting the feedstock with at least one chromatography resin having at least one ligand specific for the non-human host IgG heavy chain or light chain under conditions that allow binding of at least some of the non-human host IgG heavy chain or light chain to the chromatography resin having the ligand. Desirably, the ligand is a VHH ligand, which can, if desired, be prepared by the methods described in U.S. Patent Application Publication No. 20030078402, and U.S. Pat. Nos. 6,399,763 and 6,670,453. The chromatography resin can be any of the resins described herein. Desirably, the chromatography resin is suitable for the immobilization of a small ligand (e.g., the resin NHS-activated Sepharose™ 4 Fast Flow (activated with 6-aminohexanoic acid to form active N-hydroxysuccinimide esters; Amersham Biosciences). The flow-thru from the affinity chromatography resin contains the purified human IgG while the non-human host IgG and chimeric human/non-human host IgG is bound to the ligand of the affinity chromatography resin. The non-human host can be an ungulate, mouse, horse, pig, rat, or rabbit. Preferred ungulates are ovine, bovine, porcine, and caprine.

Preferably, this method yields a preparation of IgG that is at least 80%, 85%, 90%, 95%, or 99% or more pure. Desirably, the preparation of IgG has less than 5%, 4%, 3%, 2%, 1% or 0.5% of non-human IgG, less than 45%, 40%, 35%, 30%, 25% or 20% chimeric IgG, less than 100, 50, or 10 ppm bovine serum albumin, less than 500, 250, or 100 ppm host contaminating proteins, or less than 5, 4, 3, 2, or 1 ppm DNA, or undetectable levels of transmissible spongiform encephalopathy, viral DNA, or viral particles as detected by standard methods known in the art, such as Western blot analysis, infectivity assays or PCR analysis.

Any of the above-described methods can be combined with the membrane-mediated electrophoresis method described herein to further purify the IgG.

In a seventh aspect, the invention features a preparation of purified human IgG made using a feedstock from a non-human transgenic host. Desirably, the preparation has a ratio of human IgG to host IgG of at least 2:1, 10:1, or 100:1. In preferred embodiments, the preparation can be prepared using any of the methods of the invention. The non-human host can be an ungulate, mouse, horse, pig, rat, or rabbit. Preferred ungulates are ovine, bovine, porcine, and caprine.

In an eighth aspect, the invention features a preparation of purified human IgG made using a feedstock from a non-human transgenic host that has less than 5%, 4%, 3%, 2%, 1% or 0.5% of non-human IgG, less than 45%, 40%, 35%, 30%, 25% or 20% chimeric IgG, less than 100 or 50 ppm bovine serum albumin, less than 500, 250, or 100 ppm host contaminating proteins, or less than 5, 4, 3, 2, or 1 ppm DNA.

In a ninth aspect, the invention features a preparation of purified human IgG made using a feedstock from a non-human transgenic host that has undetectable levels of transmissible spongiform encephalopathy using standard detection methods known in the art (e.g., Western blot analysis) or undetectable viral DNA or viral particles using standard detection methods known in the art (e.g., infectivity assays or PCR analysis).

In preferred embodiments of the eighth and ninth aspects, the feedstock may be, for example, plasma, serum, ascites, or milk taken from a wild-type or transgenic mammal (e.g., ungulate, mouse, horse, pig, rat, and rabbit). Preferred ungulates are ovine, bovine, porcine, and caprine. In one embodiment, the non-human transgenic host is a transgenic bovine that produces human IgGs.

By “affinity chromatography” is meant the use of a natural or synthetic compound that specifically binds or interacts with a desired component (e.g., immunoglobulin) that is immobilized on a support or resin for the purpose of isolating, purifying, or removing the component. Chromatography reagents are known as columns or resins. An affinity chromatography resin according to the present invention binds at least 50%, 60%, 70%, 80%, 90%, 95%, or 99% of the Ig in a feedstock. Non-limiting examples of compounds used for affinity chromatography of immunoglobulin are described herein and include natural proteins such as Protein A obtained from Staphylococcus aureus, Protein G from Streptococcus sp., and Protein L from Peptostreptococcus magnus, recombinant versions thereof, or any synthetic peptide shown to specifically recognize an immunoglobulin. One example of an antibody-selective ligand that has an affinity for immunoglobulin is 4-Mercapto-Ethyl-Pyridine which is available from Ciphergen under the name MEP HyperCel™. 4-Mercapto-Ethyl-Pyridine has a hydrophobic tail and an ionizable headgroup which is uncharged and hydrophobic at physiological pH. Under acidic pH conditions, the ligand takes on a positive charge, as does the IgG, and electrostatic repulsion occurs, causing the dissociation of the IgG. Another example of an antibody selective ligand is 2-mercapto-5-benzimidazole sulfonic acid, which is available from Ciphergen under the name MBI HyperCel™. 2-mercapto-5-benzimidazole sulfonic acid has a sulfonate group present on the aromatic ring which is negatively charged over the recommended adsorption pH (5.0 to 5.5). IgG are then separated from albumin as a function of pH. IgG can then be eluted using an eluent with a basic pH.

By “caprylic acid” or “CA” is meant a carboxylic acid that is a medium-chain 8-carbon saturated fatty acid and is also known as octanoic acid. Caprylate or sodium caprylate refers to the ionized form of the of the acid and can be used as a source of CA. This form is encompassed by the term “caprylic acid” or “CA.”

By “feedstock” is meant a raw material used for chemical or biological processes.

By “immunoglobulin” or “Ig” is meant a class of proteins that act as receptors and effectors in the immune system and structurally consist of a variable region for antigen recognition, a hinge region, and a constant region for effector function. Immunoglobulins typically act as the protein mediators of humoral immunity secreted upon antigenic stimulation of B cells. There are five immunoglobulin isotypes: IgG, IgA, IgM, IgE and IgD. Of these, IgG, IgA, and IgM constitute 95% of the immunoglobulin found in serum. By “non-human immunoglobulin” is meant an immunoglobulin derived from an animal, preferably a mammal, other than a human. By “chimeric immunoglobulin” is meant an immunoglobulin that is composed of regions (e.g., variable, hinge, or constant) from at least two different species. In one example, a chimeric immunoglobulin has a heavy chain from one species (e.g., human or bovine) and a light chain from another species (e.g., human or bovine). In another example, only a portion of the heavy or light chain (e.g., variable or constant region) is from a species that is distinct from the rest of the immunoglobulin molecule. Chimeric immunoglobulin can be genetically engineered, made by mutation, or produced in a transgenic animal.

By “pH” is meant a measurement of the acidic or basic nature of a solution pH is defined as the negative logarithm of the hydrogen ion concentration in mol/L or pH=−log₁₀[H+]. A pH of about 7.0 is neutral, a pH lower than 7.0 is considered acidic, and a pH higher than 7.0 is considered basic. For purposes of the invention, a pH of 6.0 to 7.0 can be considered neutral or mildly acidic, while a pH of 7.0 to 8.5 can be considered neutral or mildly basic.

As used herein, the terms “purified” and “to purify” refer to the removal of components (e.g., contaminants, proteins, or viral particles) from a feedstock. For example, immunoglobulin can be purified by the removal of contaminating non-immunoglobulin proteins; they are also purified by the removal of immunoglobulin other than IgG. The removal of non-immunoglobulin proteins and/or the removal of immunoglobulin other than IgG results in an increase in the percent of desired IgG in the feedstock. Purity can be measured by standard assays known in the art or described herein, examples of which include SDS-PAGE followed by Coomasie blue staining as well as chromatographic methods (e.g., size exclusion chromatography (SEC) on a HPLC system). Purity of the IgG sample can be calculated from an SDS PAGE gel after scanning using a Kodak Image Station 1000 or equivalent system, or by analysis of SEC chromatogram by software on a Shimadzu HPLC system. A sample is considered pure if it is at least 90%, 95%, or 99% free of components other than the desired product (e.g., immunoglobulin).

By “membrane-mediated electrophoresis” is meant a process of separating macromolecules from complex biological samples that includes the use of membranes of selected pore sizes to separate molecules on the basis of charge or size or both. The instrument used for membrane-mediated electrophoresis typically includes a separation unit, which consists of the membranes in a cartridge formation positioned between electrodes. The membranes can be stacked to form a cartridge with multiple stream paths, which circulate in parallel. An electric field is applied across the membranes and streams, resulting in charged molecules transferring between streams towards the electrode of opposite charge. The molecular weight cut-off of the membranes and the pH of the buffer system allows for the separation of the desired macromolecules based on charge or size or both.

By “VHH ligand” is meant a single-domain heavy chain antibody, or antibody fragment, derived from camelids. In general VHH ligands have a heavy chain derived from an immunoglobulin naturally devoid of light chains that is joined together to form a multivalent single polypeptide which retains the antigen binding affinity of the parent whole immunoglobulin but which is much smaller in size and therefore less immunogenic. VHH ligands are described in detail, for example, in Frenken et al., J. Biotechnol. 78:11-21 (2000), van der Linden et al., Biochem Biophys. Acta. 1431: 37-46 (1999), Spinelli et al., Biochemistry 39:1217-1222 (2000), U.S. Patent Application Publication No. 20030078402, and U.S. Pat. Nos. 6,399,763 and 6,670,453.

Other features and advantages of the invention will be apparent from the following detailed description, and from the claims.

### BRIEF DESCRIPTION OF THE DRAWINGS

FIG. 1 is a series of graphs showing size exclusion chromatography analysis of human IgG purified by CA precipitation and a Protein G affinity column. Panel A is the CA supernatant; Panel B is the fraction eluted at pH 3.0 from the Protein G column. Each IgG sample was analyzed by size exclusion chromatography on a TSK-GEL G3000SW column connected to and controlled by a Shimadzu VP HPLC system.

FIG. 2 is a series of graphs showing size exclusion chromatography analysis of human IgG purified by CA precipitation and MEP HyperCel™ affinity column. Panel A is the CA supernatant; Panel B is the fraction eluted at pH 4.4 from the MEP HyperCel™ column. Each IgG sample was analyzed by size exclusion chromatography on a TSK-GEL G3000SW column connected to and controlled by a Shimadzu VP HPLC system.

FIG. 3 is a series of graphs showing size exclusion chromatography analysis of bovine IgG purified by CA precipitation and Protein G affinity column. Panel A is the CA supernatant; Panel B is the fraction eluted at pH 3.0 from the Protein G column. Each IgG sample was analyzed by size exclusion chromatography on a TSK-GEL G3000SW column connected to and controlled by a Shimadzu VP HPLC system.

FIG. 4 is a graph showing the elution profile of bovine IgG on a rProtein A column. Purified bovine IgG was applied onto a 5 mL rProtein A column Sepharose™ column, washed with PBS and 0.1 M sodium acetate, followed by stepwise pH washes and pH 3.0 elution. The chromatography was run by an AKTA FPLC system.

FIG. 5 is a graph showing the elution profile of human IgG on an rProtein A column. Purified human IgG was applied onto a 5 mL rProtein A-Sepharose™ column, washed with PBS and 0.1 M sodium acetate, followed by stepwise pH washes and pH 3.0 elution. The chromatography was run by an AKTA FPLC system.

FIG. 6 is a graph showing the elution profile of transgenic bovine plasma on an rProtein A column. CA-treated transgenic bovine plasma was applied onto a 5 ml rProtein A column Sepharose™ column, washed with PBS and 0.1 M sodium acetate, followed by stepwise pH washes and pH 3.0 elution. The chromatography was run by an AKTA FPLC system.

FIG. 7 is a series of graphs showing the size exclusion chromatography analysis of bovine IgG samples. Panel A shows the CA supernatant incubated at 60° C. for 2 hours. Panel B shows the pH 4.4 elution fraction purified from heat-treated (60° C. for 2 hours) CA supernatant by MEP HyperCel™ affinity chromatography. Each IgG sample was analyzed by size exclusion chromatography on a TSK-GEL G3000SW column connected to and controlled by a Shimadzu VP HPLC system.

FIG. 8 is a series of graphs showing the size exclusion chromatography analysis of bovine IgG samples. Panel A shows the MEP HyperCel™ column flow-through of CA supernatant incubated at 60° C. for 2 hours. Panel B shows the pH 3.0 elution fraction purified from heat-treated (60° C. for 2 hours) CA supernatant by MEP HyperCel affinity chromatography. Each IgG sample was analyzed by size exclusion chromatography on a TSK-GEL G3000SW column connected to and controlled by a Shimadzu VP HPLC system.

FIG. 9 is a graph showing the size exclusion chromatography analysis of bovine IgG feedstock purified from heat-treated (60° C. for 2 hours) CA supernatant by Protein G affinity chromatography. Each IgG sample was analyzed by size exclusion chromatography on a TSK-GEL G3000SW column connected to and controlled by a Shimadzu VP HPLC system.

FIG. 10 is a series of graphs showing the size exclusion chromatography analysis of human IgG samples. Panel A shows the human IVIG feedstock. Panel B shows the human IVIG purified by MEP HyperCel™ affinity chromatography, pH 4.4 elution fraction. Each IgG sample was analyzed by size exclusion chromatography on a TSK-GEL G3000SW column connected to and controlled by a Shimadzu VP HPLC system.

FIG. 11 is a series of graphs showing the size exclusion chromatography analysis of human IgG samples. Panel A shows the human IVIG incubated at 60° C. for 2 hours. Panel B shows the IgG feedstock purified from heat-treated human IVIG by MEP HyperCel™ affinity chromatography, pH 4.4 elution fraction. Each IgG sample was analyzed by size exclusion chromatography on a TSK-GEL G3000SW column connected to and controlled by a Shimadzu VP HPLC system.

FIG. 12 is a graph showing size exclusion chromatography analysis of MEP HyperCel™ column flow-through of human IVIG incubated at 60° C. for 2 hours. Each IgG sample was analyzed by size exclusion chromatography on a TSK-GEL G3000SW column connected to and controlled by a Shimadzu VP HPLC system.

FIG. 13 is a series of graphs showing size exclusion chromatography analysis of human IgG samples. Panel A: CA supernatant incubated at 60° C. for 2 hours; Panel B: IgG feedstock purified from heat-treated CA supernatant by MEP HyperCel affinity chromatography, pH 4.4 elution fraction. Each IgG sample was analyzed by size exclusion chromatography on a TSK-GEL G3000SW column connected to and controlled by a Shimadzu VP HPLC system.

FIG. 14 is a graph showing size exclusion chromatography analysis of human IgG feedstock purified from heat-treated (60° C. for 2 hours) CA supernatant by Protein G affinity chromatography. The IgG sample was analyzed by size exclusion chromatography on a TSK-GEL G3000SW column connected to and controlled by a Shimadzu VP HPLC system.

FIG. 15 is a graph showing size exclusion chromatography analysis of human IgG feedstock purified from heat-treated (60° C. for 2 hours) CA supernatant by Protein A affinity chromatography. The IgG sample was analyzed by size exclusion chromatography on a TSK-GEL G3000SW column connected to and controlled by a Shimadzu VP HPLC system.

FIG. 16 is a schematic showing four types of IgGs present in transgenic bovine plasma.

FIG. 17 is a graph showing the purification of human IgG by a horse anti-bovine IgG immunoaffinity column. The IgG feedstock from pH 3.0 elution off the rProtein A column was adjusted to pH 8.0 and then passed through a horse anti-bovine IgG column. The flow-through (unbound fraction) was collected as the human IgG feedstock.

FIG. 18 is a graph showing the removal of bovine IgG and chimeric IgG by a horse anti-bovine IgG immunoaffinity column. The IgG feedstock from pH 3.0 elution off the rProtein A column was adjusted to pH 8.0 and then passed through a horse anti-bovine IgG column. The flow-through (unbound fraction) was collected as the human IgG feedstock. The bound material, eluted with 50 mM glycine-HCl, pH 3.0, contains bovine IgG and chimeric IgG fraction.

FIG. 19 is an autoradiograph of a western blot showing human IgG purified from transgenic bovine plasma. Human IgG was detected with rabbit anti-human IgG HRP (Panel A), and bovine IgG was detected with sheep anti-bovine IgG HRP (Panel B). HC: IgG heavy chain, LC: IgG light chain.

FIG. 20 is an HPLC size exclusion chromatogram for the IgG sample pre Gradiflow™ system (containing IgG aggregates and BSA) and post Gradiflow™ system. Note that IgG aggregates and BSA were removed following Gradiflow™ system.

### DETAILED DESCRIPTION OF THE INVENTION

We have discovered improved methods for purifying immunoglobulin from wild type or transgenic animal fluids such as plasma, sera, or milk or from cell culture supernatants containing polyclonal or monoclonal antibodies. These methods provide significant improvement over the art in terms of efficiency, yield, and purity of immunoglobulins. These methods are particularly useful for purifying human IgG, with minimal contamination of non-IgG proteins, bovine serum albumin (BSA), host-contaminating proteins, viral DNA, viral particles, transmissible spongiform encephalopathy (TSE), IgG dimers, or IgG aggregates. Human IgG purified by the methods described herein can be used for therapeutic, diagnostic, or research purposes. Three of the methods described are designed to purify human IgG produced in a transgenic host through specific removal of the non-human host proteins, thus producing a purified human IgG with low levels, if any, of contaminating host IgG. The methods can also be tailored for the specific removal of IgG dimers and aggregates, resulting in a highly purified preparation of IgG that is useful for therapeutic, diagnostic or research purposes. Each of the methods used alone is effective for the purification of IgG but, if desired, can also be used in combination with any of the additional methods (or part thereof) described herein for additional purification of IgG.

The methods can be generally summarized as follows:

(I) A method for purifying IgG using a mono or polyalkanoic acid (e.g., CA) as a precipitant, followed by chromatography using a resin with an affinity for IgG.

(II) A method for purifying IgG using a mono or polyalkanoic acid (e.g., CA) as a precipitant, followed by membrane-mediated electrophoresis to separate the purified IgG.

(III) A method for purifying human IgG from a feedstock that contains human and non-human IgG using affinity chromatography followed by stepwise washes with buffers that increase with acidity in each step to separate the non-human or chimeric IgG from the human IgG.

(IV) A method for purifying IgG monomers and removing or reducing IgG dimers and/or IgG aggregates using differential pH washes and a chromatography resin that has an affinity for IgG monomers.

(V) A method for purifying human IgG from a non-human host feedstock that contains human, non-human, and/or chimeric IgG using an anti-human IgG affinity chromatography resin followed by washes with a buffer that causes the dissociation of the non-human IgG to separate the non-human or chimeric IgG from the human IgG and then further removing the non-human or chimeric IgG using an anti-host IgG affinity chromatography resin (e.g., a resin having a VHH ligand). Alternatively, the affinity chromatography steps may be reversed to first remove the non-human IgG using an anti-host IgG affinity chromatography resin (e.g., a resin having a VHH ligand) and then to further purify the human IgG using an anti-human IgG affinity chromatography resin followed by elution of the human IgG from the anti-human IgG affinity chromatography resin.

(VI) A method for purifying human IgG from a feedstock that contains human and non-human IgG using an anti-host IgG ligand, for example, a VHH ligand.

Any of the above methods can be used either alone or in any combination. The combination of the methods need not include every step of each of the methods and can include any combination of any steps of any of the methods described herein.

The following general parameters are useful for practicing the methods of the invention. These are provided as guidance and should not be construed as limiting.

Feedstocks

The methods described herein for purifying immunoglobulin can be used on feedstocks derived from any animal, including wild-type and transgenic animals. Preferred feedstocks include any bodily fluid such as plasma, serum, milk, ascites, or other IgG containing sources such as Cohn fractions. Preferred animals include mammals, e.g., ungulate, human, mouse, horse, pig, rat, and rabbit. Preferred ungulates are ovine, bovine, porcine, and caprine. Preferably, the animal is a transgenic animal that can be used to produce human IgG. The most preferred feedstock is plasma or serum from a transgenic bovine that produces human IgG.

The methods of the invention can also be used to purify IgG from a cell culture supernatant (e.g., monoclonal IgG from a hybridoma cell line) or to further purify desired IgG from a sample of immunoglobulin, such as IVIG.

The methods of the invention are particularly useful for the removal of undesired IgG aggregates, IgG from the host species, BSA, fetal calf serum, host contaminating proteins, viruses, and TSE.

Reaction Conditions

We have discovered that combining the use of high concentrations of mono or polyalkanoic acids (e.g., CA) as a precipitant under low pH conditions followed by an affinity chromatography step allows for efficient purification of IgG from a variety of feedstock such as animal plasma or serum and results in a high yield of purified IgG. For CA precipitation, the feedstock is generally adjusted to a pH of about 4.0 to 5.0. The feedstock can be diluted in any appropriate buffer (e.g., a buffer containing Na-acetate, pH 4.0) followed by an adjustment of the sample to the desired pH. Feedstocks can also be undiluted and the pH of the feedstock can be adjusted directly.

Suitable mono or polyalkanoic acids include any alkanoic acid generally having the formula CₙH₂O₂ and having from 4 to 12 carbon atoms, preferably from 6 to 9 carbon atoms. Non-limiting examples are pentanoic acid, hexanoic acid, heptanoic acid, octanoic acid (also known as CA), nonaoic acid, decanoic acid, (z)-hex-2-enoic acid, 6-methylheptanoic acid, 3-chloropentanoic acid, hexanedioic acid, 6-hydroxy-4-oxononaoic acid. Desirably, the alkanoic acid is CA. Although unbranched alkanoic acids are preferred, branched alkanoic acids can also be used.

The alkanoic acid (e.g., CA) used for the precipitation steps in the methods of the invention can range in concentration from 3% to 10%. Preferred concentrations include 3%, 3.5%, 4%, 4.5%, 5%, 5.5%, 6%, 6.5%, 7%, 7.5%, 8%, 8.5%, 9%, 9.5%, and 10%. The concentration of alkanoic acid (e.g., CA) used for the precipitation steps in the methods of the invention can also be determined by calculating the total protein concentration of the feedstock and adding an amount of alkanoic acid (e.g., CA) sufficient to achieve a ratio of alkanoic acid/total protein equal to 0.75 to 2.25, preferably 1 to 2.25.

After CA precipitation, the precipitate is removed from the supernatant using standard techniques known in the art such as centrifugation or filtration. In one example, the precipitated material can be removed by centrifugation at 6000 rpm and 20° C. for 30 minutes using a GS3 or GSA rotor with a Sorvall RC-5B centrifuge. In another example the precipitated material can be removed by filtration with a depth filter device from Pall Life Sciences and filter aid such as Celpure from Advanced Minerals Corp. After removal of the precipitate, the pH of the supernatant can be adjusted to a range that is optimal for subsequent affinity chromatography. The pH range will depend on the specific requirements of the affinity chromatography resin used, although the pH range is typically about pH 5.0 to 8.5 (e.g., pH 5.0, 5.5, 6.0, 6.5, 7.0, 7.5, 8.0, or 8.5).

Additional filtration or centrifugation steps can be included after adjusting the pH to remove any additional precipitate. Non-limiting examples of filters useful for removal of BSA, host contaminating proteins, and viruses are depth filters, which are typically characterized by their design to retain particles within a filter matrix. Non-limiting examples of filter aids, which are inorganic mineral powders or organic fibrous materials used in combination with filtration hardware to enhance filtration performance, include diatomite, perlite, and cellulose. One preferred example of a filter aid useful in the methods of the invention is Celpure 1000 (Advanced Minerals Corporation).

After filtration or centrifugation, the supernatant is applied to an affinity chromatography resin which contains a ligand with an affinity for IgG covalently bound to a solid support (see below). The resin is prepared as described by the manufacturer's instructions and, after addition of the supernatant, the resin is washed as described by the manufacturer's instructions for the particular ligand/resin used, or in the case of specific methods described herein, washed using a series of low pH buffers. We have discovered that the use of low pH buffer washes is an effective method for dissociating the bovine IgG from a Protein A resin without significantly dissociating the human IgG bound to the resin. The pH range of the low pH buffers can range from about 4.0 to 7.0 and is preferably pH 4.0, 4.2, 4.4, 4.46, 4.5, 4.6, 4.7, 4.8, 4.9, 5.0, 5.2, 5.5, 6.0, 6.5, 7.0. For the low pH buffers, the resin is preferably washed with buffers of decreasing pH (e.g., pH 5.2, then pH 4.8, then pH 4.46).

After washing, the IgG is then eluted from the affinity chromatography resin using an elution buffer that is appropriate for the particular affinity chromatography resin used. In one example, IgG is eluted from a Protein A-based resin using a buffer with a pH of 3.0. In another example, the IgG is eluted from a MEP HyperCel™ resin using a buffer with a pH of 3.0 to 4.5. In a preferred embodiment, the pH of the elution buffer used for the MEP HyperCel™ resin is 4.4.

For the methods that relate to the removal of non-human IgG and human/non-human chimeric IgG using anti-non-human host affinity chromatography (e.g., methods #5 and #6, above), the pH of the eluate from the first affinity chromatography resin is adjusted to a pH 7.0 to 8.5, most preferably 7.0, 7.5, 8.0, or 8.5. The pH adjusted eluate is then added to an anti-host IgG-based resin. The flow-through contains the human IgG while the non-human and chimeric IgG remain bound to the resin and can be eluted by acidic buffers, if desired, as described above.

For the methods that relate to the removal of non-human IgG and human/non-human chimeric IgG using ligands that are specific for the non-human host (e.g., methods #5 and #6, above), ligands are immobilized on a matrix and used to selectively bind to and remove the non-human or human/non-human chimeric IgG. This approach requires the use of ligands specific for the non-human IgG heavy chain or light chain with minimal or no cross-reactivity with fully human IgG heavy chain or light chain domains. Exemplary ligands are VHH ligands, for example, produced by the methods of U.S. Patent Application Publication No. 20030078402, and U.S. Pat. Nos. 6,399,763 and 6,670,453. These patents describe phage display library screening technology using the variable regions of camelid (camel and llama) antibodies. Camelid antibodies are unusual in that one class has no light chain at all and all of the antigen binding diversity can be captured in a phage display library of the heavy chain variable regions alone (called VHH domains). The resulting small single chain proteins (˜12 kD), known as VHH ligands, function very similarly to the whole antibodies in terms of binding characteristics. The VHH ligands are then immobilized on an affinity chromatography resin, such as any of the chromatography resins described below, and used to remove the non-human host or human/non-human host chimeric IgG. One preferred chromatography resin is NHS-activated Sepharose™ 4 Fast Flow.

The buffers used in the methods of the invention are prepared using standard methods and reagents known in the art. Non-limiting examples of buffer components include acetic acid, sodium acetate, sodium chloride, Tris HCl, Tris Base, glycine, sodium carbonate, and sodium phosphate (see Sambrook, Fritsch, and Maniatis (1989) Molecular Cloning, Cold Spring Harbor Laboratory Press for a general list of buffers and buffer components).

The yield and purity of the purified IgG samples can be measured using assays known in the art. Non-limiting examples of methods for determining protein purity are Western blot analysis, size exclusion chromatography, SDS-PAGE separation followed by staining with Coomassie blue or silver staining. The yield is determined using assays known in the art. Non-limiting examples of methods for determining protein yield can be found, for example, in McKinney and Parkinson, supra.

Chromatography

Chromatography, particularly affinity chromatography, can be used to further purify the desired IgG. Preferred affinity chromatography resins will include any ligand or compound capable of binding non-covalently to at least one IgG, or a portion of an IgG, the ligand or compound being immobilized on a chromatography support.

Ligands can be naturally occurring proteins, recombinant forms of naturally occurring proteins, synthetic proteins, recombinant proteins, or compounds with a specific affinity for at least a portion of IgG. The following is a list of preferred ligands used for affinity chromatography of IgG. This list is provided by way of example and is not intended to limit the invention in any way.

Protein A, which is derived from the bacterium Staphylococcus aureus, has a strong and specific affinity for the Fc fragment of IgG and has been used as an affinity ligand for purifying IgG. Protein A can be immobilized on a large variety of solid support materials, such as chromatographic beads and membranes.

Protein G has similarly been used as an affinity ligand. See Bjorck et al., J. Immunol., 133:969 (1984). Protein G also interacts with the Fc fragment of immunoglobulin and is particularly effective in isolating IgG antibodies of class 1.

Recently, a third protein has been identified as an effective affinity ligand for purifying immunoglobulin. This protein is Protein L from Peptostreptococcus magnus. Protein L, as contrasted with Protein A and Protein G, interacts specifically with the light chains of IgG antibodies without interfering with their antigen binding sites. This specificity permits Protein L to complex not only with antibodies of the IgG class but also with antibodies of the IgA and IgM classes.

Anti-antibodies represent still another type of affinity ligand used for Ig purification.

In addition to the above-described protein-based affinity ligands, there are numerous lower molecular weight pseudobioaffinity (i.e., less specific) ligands which have been used for immunoglobulin purification. Histidine, pyridine, and related compounds represent one type of pseudobioaffinity ligand commonly used for antibody purification. See for example, Hu et al., J. Chromatogr. 646:31-35 (1993); El-Kak et al., J. Chromatogr. 604:29-37 (1992); Wu et al., J. Chromatogr., 584:35-41 (1992); El-Kak et al., J. Chromatogr. 570:29-41 (1991); and U.S. Pat. Nos. 5,185,313; 5,141,966; 4,701,500; and 4,381,239.

Thiophilic compounds represent another class of pseudobioaffinity ligands. An adsorbent utilizing one type of thiophilic compound is disclosed by Porath et al., FEBS Lett. 185:306 (1985). This type of adsorbent is produced by reacting either a hydroxyl- or thiol-containing support first with divinyl sulfone and then with mercaptoethanol. The aforementioned adsorbent utilizes a salt-promoted approach to adsorb immunoglobulin. Elution of adsorbed immunoglobulin is effected by decreasing salt concentration and/or by modifying pH.

Another type of pseudobioaffinity adsorbent capable of adsorbing antibodies utilizes mercaptopyridine as its ligand. See Oscarsson et al., J. Chromatogr. 499:235-247 (1990). This type of adsorbent is generated, for example, by reacting mercaptopyridine with a properly activated solid support. The adsorbent thus formed is capable of adsorbing antibodies under high salt conditions.

One example of a pseudoaffinity adsorbent capable of adsorbing antibodies utilizes 4-mercapto-ethyl-pyridine as its ligand and can also include a cellulose support (e.g., MEP HyperCel™).

Other pseudobioaffinity adsorbents utilizing thiophilic compounds are described in the following patents and publications, all of which are incorporated herein by reference: U.S. Pat. No. 4,897,467; published EP00168363; Oscarsson et al., J. Immunol. Methods 143:143-149 (1991); and Porath et al., Makromol. Chem., Macromol. Symp. 17:359-371 (1988).

Another group of low molecular weight ligands capable of selectively binding immunoglobulin includes pentafluoropyridine and N-dimethylaminopyridine reacted with ethylene glycol, glycine or mercaptoethanol. See Ngo, J. Chromatogr. 510:281 (1990), which is incorporated herein by reference. Adsorbents utilizing these materials can be used to isolate immunoglobulin in either high salt or low salt buffers or to isolate other types of proteins under low salt conditions. Elution of adsorbed proteins can be obtained by lowering pH.

Still other low molecular weight pseudobioaffinity ligands have been identified as being capable of selectively binding antibodies from egg yolk and other biological liquids. These ligands are special dyes. Elution of the bound antibodies from the ligands is achieved by special displacers.

Additional examples of suitable ligands that can be used for affinity chromatography are found in U.S. Pat. Nos. 6,207,807 and 6,610,630.

Another desired group of ligands is the VHH ligand specific for the non-human IgG heavy chain or light chain with minimal or no cross-reactivity with fully human IgG heavy chain or light chain domains. These ligands are described in detail above.

For immobilization of the antibody-specific ligand, any number of different solid supports may be utilized. For example, the solid support material may be composed of polysaccharides, such as cellulose, starch, dextran, agar or agarose, or hydrophilic synthetic polymers, such as substituted or unsubstituted polyacrylamides, polymethacrylamides, polyacrylates, polymethacrylates, polyvinyl hydrophilic polymers, polystyrene, polysulfone or the like. Other suitable materials for use as the solid support material include porous mineral materials, such as silica, alumina, titania oxide, zirconia oxide and other ceramic structures. Alternatively, composite materials may be used as the solid support material. Such composite materials may be formed by the copolymerization of or by an interpenetrated network of two or more of the above-mentioned entities. Examples of suitable composite materials include polysaccharide-synthetic polymers and/or polysaccharide-mineral structures and/or synthetic polymer-mineral structures, such as are disclosed in U.S. Pat. Nos. 5,268,097; 5,234,991; and 5,075,371.

The solid support material of the present invention may take the form of beads or irregular particles ranging in size from about 0.1 mm to 1000 mm in diameter, fibers (hollow or otherwise) of any size, membranes, flat surfaces ranging in thickness from about 0.1 mm to 1 mm thick, and sponge-like materials with holes from a μm to several mm in diameter.

Preferably, the ligands described above are chemically immobilized on the solid support material via a covalent bond formed between the mercapto group of the ligand and a reactive group present on the solid support. Reactive groups capable of reacting with the mercapto group of the present ligand include epoxy groups, tosylates, tresylates, halides and vinyl groups. Because many of the aforementioned solid support materials do not include one of the reactive groups recited above, bifunctional activating agents capable of both reacting with the solid support materials and providing the necessary reactive groups may be used. Examples of suitable activating agents include epichlorhydrin, epibromhydrin, dibromo- and dichloropropanol, dibromobutane, ethyleneglycol diglycidylether, butanediol diglycidylether, divinyl sulfone and the like.

Typical examples of suitable supports are Sepharose™, agarose, the resin activated-CH Sepharose™ 4B (N-hydroxysuccinimide containing agarose) from Pharmacia (Sweden), the resin NHS-activated Sepharose™ 4 Fast Flow (activated with 6-aminohexanoic acid to form active N-hydroxysuccinimide esters; Amersham Biosciences), the resin CNBr-activated Sepahrose™ Fast Flow (activated with cyanogen bromide; Amersham Biosciences) the resin PROTEIN PAK™ epoxy-activated affinity resin (Waters, USA), the resin EUPERGIT™ C30 N (Rohm & Haas, Germany), UltraLink Biosupport Medium (Pierce), Trisacryl GF-2000 (Pierce), or AFFI-GEL™ from BioRad (USA). Preferably, the support for affinity chromatography is preactivated with epoxyde groups for direct coupling to peptides and proteins.

The affinity chromatography resins useful for practicing the methods of the invention include, but are not limited to, any combination of ligand or compound described above with any of the supports described above. Non-limiting examples of specific affinity chromatography resins are Protein A-Sepharose™, Protein A-agarose, Protein A-agarose CL-4B, Protein G-Sepharose™, Protein G-agarose, Protein G-agarose CL-4B, Protein L-agarose, Protein A/G agarose (various versions of all of the above are available from various manufacturers, e.g., Sigma-Aldrich, Amersham, and Pierce), KAPTIV™ immunoaffinity matrices (e.g., KAPTIV-GY™, KAPTIV-AE™, KAPTIV-M™, all from Tecnogen Inc.), Cellthru BigBead™ (Sterogene), Protein A Ultraflow™ (Sterogene), Protein A Cellthru™ 300 (Sterogene), QuickMab (Sterogene), QuickProtein A™ (Sterogene), Thruput™ and Thruput Plus (Sterogene), PROSEP-A or PROSEP-G (Millipore), MEP Hypercel™ (Ciphergen), MBI Hypercel™ (Ciphergen) and CM Hyperz™ (Ciphergen), and any variations of the above.

The methods used for the affinity chromatography depend on the specific reagent used and are typically supplied by the manufacturer or known in the art (see for example Harlow and Lane, Antibodies: A Laboratory Manual, Cold Spring Harbor Laboratory, 1988).

Generally, the affinity chromatography reagent is packed in a chromatographic column, equilibrated with a buffer capable of promoting an interaction between immunoglobulin and the affinity ligand, and then contacted with a feedstock, supernatant, or sample comprising at least one immunoglobulin. The column is then washed with at least one liquid capable of eluting the impurities without interfering with the interaction between immunoglobulin and the affinity ligand, and the immunoglobulin is then eluted using an eluent.

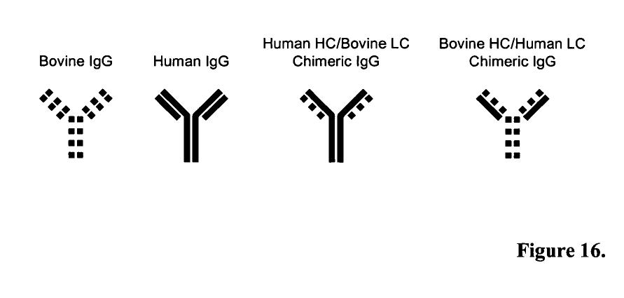

In one particular example, non-human IgG can be removed from a feedstock, supernatant, or sample containing both human and non-human IgG using a ligand with a specific affinity for the non-human IgG. For this particular example, a feedstock taken from a transgenic bovine animal, containing bovine IgG, chimeric IgG, and human IgG (FIG. 16), is treated or manipulated for purification of the IgG-containing fraction (e.g., CA precipitation and affinity chromatography as described above), and the eluate containing all the IgG is then applied to an anti-bovine IgG-Sepharose™ resin. The flow-through in this example contains the human IgG, while the bovine and chimeric IgG remain on the resin and can be eluted separately, if desired.

Membrane-Based Electrophoresis

The IgG obtained from any of the methods described herein can be further purified using membrane-mediated electrophoresis.

Membrane-mediated preparative electrophoresis technologies have been developed to purify macromolecules from complex biological samples. Generally, the membrane-mediated electrophoresis technology employs a cartridge with a stacked polyacrylamide gel membrane which has little or no hydraulic permeability, but which allow electrophoretic transport of proteins below a controllable size range. Upstream and downstream flow channels are formed, separated by one membrane, and separated from the electrodes and electrode buffer channels by the other membranes. The feedstream (at a controlled pH) is recirculated with a pump through the one side and the product is recirculated by a second pump through the other side (residence times in the cartridge of 1-2 seconds). The various flow streams are cooled to remove the heat produced by the electric current. Conditions are set up so that the sample is appropriately charged so that it moves across the separation membrane from the feedstream to the product channel. Molecular charge, generated by the choice of suitable buffer pH systems, is employed in combination with membranes of selected pore sizes to separate molecules on the basis of charge and/or size. Target molecules, such as IgG, can be purified under conditions where the IgG is in a native or denatured state.

One preferred example of an instrument useful for membrane-mediated electrophoresis is the Gradiflow™ instrument. Gradiflow™ has a separation unit, consisting of three molecular weight cutoff membranes in a cartridge formation positioned between electrodes. The membranes are stacked to form a cartridge with multiple stream paths, which circulate in parallel. An electric field is applied across the membranes and streams, resulting in charged molecules transferring between streams towards the electrode of opposite charge. Molecular weight cut-off (MWCO) of membranes provides the selective means for size separations.

The appropriate pH and running time for membrane-mediated electrophoresis can be determined by the skilled artisan using the manufacturer's instructions. In one example, the IgG sample is dialyzed against a buffer with a pH of 5.0 and then run on a Gradiflow™ system for 6 to 24 hours. In another example, the pH of the sample containing the IgG is adjusted to a neutral pH, the supernatant is heated for a time and to a temperature (e.g., 60° C.) sufficient to form IgG aggregates. Bovine serum albumin (BSA) is then added and the supernatant/BSA mixture is dialyzed against an appropriate buffer having a pH of 5.0. The IgG is then separated from the supernatant and BSA using membrane-mediated electrophoresis.

Techniques for carrying out each method of the invention are now described in detail, using particular examples. These examples are provided for the purpose of illustrating the invention, and should not be construed as limiting.

### EXAMPLES

### Example 1

### Purification of IgG by Caprylic Acid Precipitation and Affinity Chromatography

In a first purification technique, human plasma or bovine (wild type or transgenic) plasma was pH adjusted to 4.5 with the addition of 15% acetic acid, and then treated directly with 6% (v/v) CA at pH 4.5 for 30 minutes at 20-25° C., with constant stirring. The feedstock was then centrifuged at 6,000 rpm with a GSA rotor at room temperature. The insoluble material was discarded, and the pH of the supernatant was adjusted to approximately pH 7.5 to 8.0 with addition of 1M Tris or 1N NaOH. The pH-adjusted feedstock was then filtered through a 0.22 micron filter and applied to IgG affinity resins such as Protein A Sepharose™, Protein G Sepharose™, or MEP HyperCel™. After washing with PBS or 20-50 mM Tris-HCl, pH 7.5 to 8.5 in the presence or absence of 0.15 M NaCl, IgG was eluted using low pH buffers. 50 mM glycine-HCl, pH 3.0 buffer was used for Protein A and Protein G resins, while 50 mM sodium acetate, pH 4.4 was used for MEP HyperCel™ column.

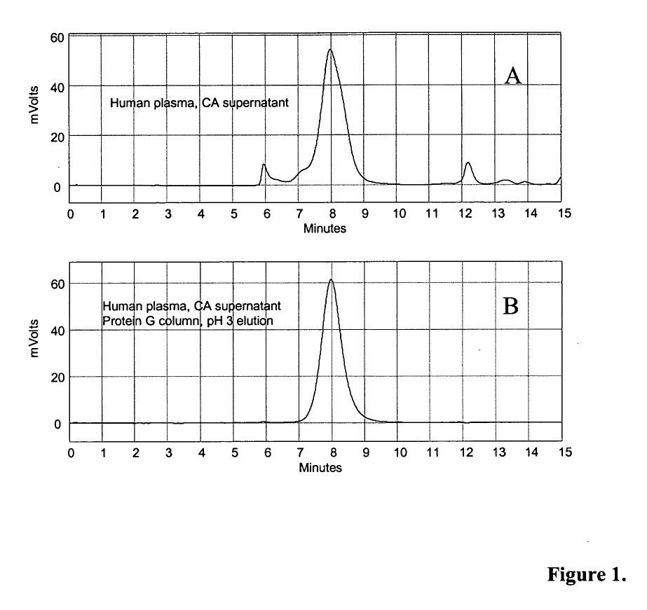

FIG. 1 shows the size exclusion chromatography (SEC) result of human IgG purified by CA precipitation and Protein G affinity chromatography. SEC was analyzed on a TSK-GEL G3000SW column connected to and controlled by a Shimadzu VP HPLC system. The protein peak at retention time of approximately 8 minutes indicated the purity of IgG feedstock eluted from the Protein G column.

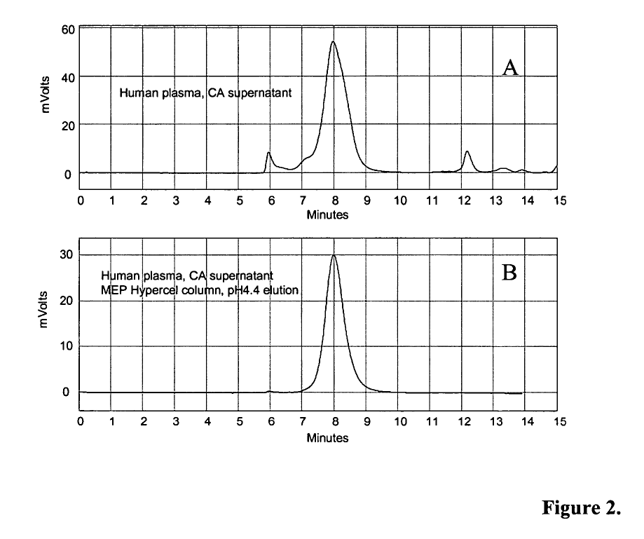

FIG. 2 shows the SEC result of human IgG purified by CA precipitation and MEP HyperCel™ affinity chromatography. SEC was analyzed on a TSK-GEL G3000SW column connected to and controlled by a Shimadzu VP HPLC system. The purified protein has a peak at retention time of approximately 8 minutes, indicating the high purity of IgG feedstock eluted from MEP HyperCel™ column.

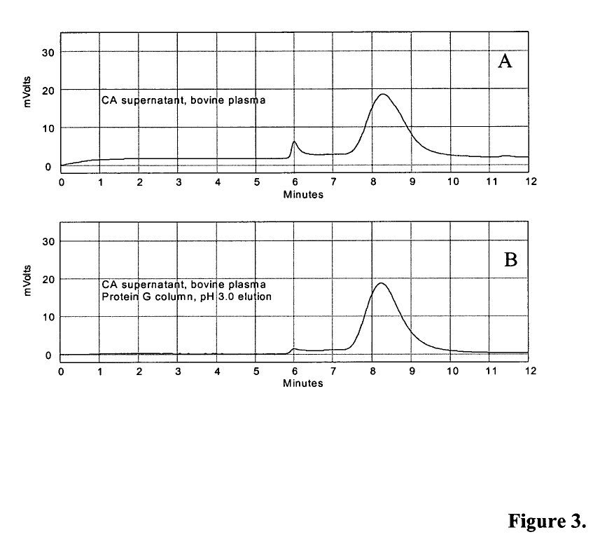

FIG. 3 shows the SEC result of bovine IgG purified by CA precipitation and Protein G affinity chromatography. SEC was analyzed by size exclusion chromatography on a TSK-GEL G3000SW column connected to and controlled by a Shimadzu VP HPLC system. The purified protein has a peak at retention time of approximately 8 minutes, indicating the high purity of IgG feedstock eluted from the Protein G column.

In addition to the high IgG purity following CA precipitation and affinity chromatography, this combination purification method also generated an IgG product that contains no detectable level of BSA. Table 1 shows the BSA concentrations in the feedstocks pre- and post-CA treatment, and post-Protein G column purification as measured by an ELISA kit from Cygnus. CA treatment of bovine plasma reduced the concentration of BSA by approximately 23,000 fold, and further purification by Protein G affinity column reduced the BSA to a level below detection.

TABLE 1
BSA levels in Bovine IgG feedstocks Determined by ELISA

| Feedstocks | BSA Concentration |
|---|---|
| Bovine Plasma | 23.25 mg/ml |
| Protein G purified feedstock from bovine | 8.8 μg/ml |
| plasma | |
| CA supernatant of bovine plasma | 930 ng/ml |
| Protein G purified feedstock from CA | Below detection limit |
| supernatant | |

### Example 2

### Purification of IgG by Caprylic Acid Precipitation and Affinity Chromatography Using CA/Total Protein Ratios

In order to get consistent recovery of IgG and removal of other contaminating proteins, the amount of CA added into a feedstock was calculated based on total protein. In this example, total protein concentration was measured by conventional protein assay methods and CA was added to achieve to ratio of CA/total protein of 0.75 to 2.25.

Table 2 shows the total protein recovery and BSA concentrations in the feedstocks pre- and post-CA treatment as measured by an ELISA kit from Cygnus. For CA precipitation with undiluted bovine plasma, the pH of the plasma was adjusted to 4.8, and CA was added to achieve a ratio of CA/total protein as shown and mixed vigorously. For CA precipitation with diluted plasmas, the plasma was diluted with appropriate amount of 60 mM Na-acetate, pH 4.0, followed by an adjustment to a final pH of 4.8. In both cases, samples were incubated at room temperature overnight, followed by centrifugation to collect the supernatant, which was then measured for total protein and BSA concentrations.

TABLE 2
Effect of CA/Protein Ratios on Protein (IgG) Recovery and BSA
Levels following CA precipitation.

| | | Total protein (mg) | |
|---|---|---|---|
| | CA/protein | recovered from per | BSA (μg/ml) in |
| Sample ID | ratio | ml starting plasma | CA supernatant |
| Undiluted Plasma | | | |
| 1 | 0.75 | 11.2 | 6.68 |
| 2 | 1.0 | 9.6 | 1.34 |
| 3 | 1.75 | 7.8 | 0.0 |
| 4 | 2.25 | 7.1 | 0.252 |
| 1:2 diluted plasma | | | |
| 5 | 1.0 | 11.9 | 3.38 |
| 6 | 2.25 | 9.2 | 0.218 |

Table 3 shows the total protein recovery and BSA concentrations in the feedstocks pre- and post-CA treatment as above except samples were incubated at room temperature for 30 minutes, followed by centrifugation to collect the supernatants.

TABLE 3
Effect of CA/Protein Ratios and Plasma Dilutions (Protein
Concentrations) on Protein (IgG) Recovery and BSA

| | | Total protein (mg) | |
|---|---|---|---|
| | CA/protein | recovered from per | BSA (μg/ml) in |
| Sample ID | ratio | ml starting plasma | CA supernatant |
| Undiluted Plasma | | | |
| 1 | 1.0 | 15.3 | 0.16 |
| 2 | 1.25 | 14.5 | 0.0 |
| 1:2 diluted plasma | | | |
| 3 | 1.0 | 16.5 | 18.35 |
| 4 | 1.25 | 15.6 | 0.08 |
| 1:1 diluted plasma | | | |
| 5 | 1.0 | 16.1 | 0.033 |
| 6 | 1.25 | 16.0 | 0.0 |

Table 4 shows the total protein recovery, bovine IgG concentration, BSA concentration, and the host contaminating protein (HCP) concentration from various bovine plasma feedstock pre- and post-CA treatment as described above. BSA and HCP were measured by an ELISA kit obtained from Cygnus, N.C.

TABLE 4
Removal of BSA and HCP by Caprylic Acid Fractionation in Various
Bovine Plasmas (plasma diluted 1:1, CA added at CA/protein = 1:1 ratio).

| | Bovine | | BSA in | HCP in | | |
|---|---|---|---|---|---|---|
| | IgG, | Total | plasma, | plasma, | HCP in CA | BSA in CA |
| Ani- | mg/ml | Plasma | mg/ml | mg/ml, | supernatant | supernatant |
| mal | by | Protein, | (by | by | by ELISA, | by ELISA, |
| ID | ELISA | mg/ml | HPLC) | ELISA | μg/ml | μg/ml |
| 1206 | ND | 50.0 | ˜40 | 12-15 | 10.9 | ND |
| 608 | 14.5 | 55.73 | 33.3 | ND | 4.9 | 9.1 |
| 607 | 11.8 | 61.31 | 36 | ND | 6.4 | 3.1 |
| 565 | 10.3 | 63.51 | 44.2 | ND | 4.8 | 0.01 |
| 564 | 7.9 | 68.42 | 46.9 | ND | 4.2 | 8.8 |
| 554 | 27.2 | 78.96 | 45.1 | ND | 7.7 | 21.6 |
| 540 | 12.5 | 70.76 | 42.9 | ND | 2.9 | 0.11 |

ND: not determined.

These results demonstrate the effectiveness of CA when added in an amount sufficient to establish a ratio of CA/total protein of 0.75 to 2.25. Precipitation of bovine plasma with CA can reduce the level of HCP (in this case, bovine plasma proteins other than IgG and BSA) from 10-15 mg/ml to a few micrograms/ml. Importantly, the CA precipitation method is robust for a variety of plasmas with different concentrations of total proteins, BSA, and IgG. In addition to BSA and HCP clearance, CA precipitation was also effective in inactivating enveloped viruses (e.g., TSE and BSE, which are the agents that cause mad cow disease), when combined with a depth filter and a filter aid (such as Celpure P1000 from Advanced Minerals Corporation.

The BSA and HCP in CA supernatant can be further reduced, thereby enhancing the purity of the desired IgG, by affinity chromatography on columns such as Protein G and MEP HyperCel. Table 5 shows the BSA and HCP concentrations after CA precipitation of bovine plasma followed by affinity chromatography columns using standard protocols. Table 6 shows the BSA and HCP concentrations after CA precipitation of human IgG spiked, bovine IgG deficient bovine plasma followed by affinity chromatography columns using standard protocols. For this sample, bovine plasma was passed through a MEP HyperCel column and flow-thru (non-binding fraction) was collected. The flow-thru is deficient in bovine IgG. Purified human IgG was added to the flow-thru sample to a final concentration of 2.5 mg/ml. CA was added per the method described above.

TABLE 5
Clearance of BSA and HCP by affinity chromatography.

| Affinity Column | BSA, ppm | HCP, ppm |
|---|---|---|
| Pre-affinity column | 47.2 | 1817 |
| Post-Protein G column | 9.5 | 23 |
| Post MEP HyperCel column | 10.1 | 48.5 |

TABLE 6
Clearance of BSA and HCP by affinity chromatography.

| Affinity Column | BSA, ppm | HCP, ppm |
|---|---|---|
| Pre-affinity column | 82 | 28502 |
| Post-Protein A column | 8.4 | 56.4 |
| Post anti-human IgG column | 6.2 | 114.0 |

### Example 3

### The use of rProtein A Chromatography in Combination with Low pH Washes to Separate Human IgG from Bovine IgG

In another technique, purified bovine IgG (purified from bovine plasma through a 5 ml Protein G Sepharose™ column) or purified human IgG (purchased from Bethyl Lab) was applied onto a 5 ml HiTrap rProtein A Sepharose™ column (from Amersham) which had been equilibrated with 25 ml of phosphate-buffered saline (PBS). rProtein A is a recombinant version of Protein A. Following feedstock application, the column was washed with approximately four bed volumes of PBS and one bed volume of 0.1 M sodium acetate until A₂₈₀ baseline was reached. The column was then stepwise washed with 10 column volumes of each of the following buffers with different pH values: pH 5.20, 4.80, and 4.46. Each low pH buffer was prepared by mixing different portions of 0.1 M sodium acetate and 0.1 M acetic acid. For example, mixing two parts of 0.1 M acetic acid with eight parts of 0.1 M sodium acetate results in a buffer with pH 5.20; four parts of 0.1 M acetic acid with six parts of 0.1 M sodium acetate results in a buffer with pH 4.80; six parts of 0.1 M acetic acid with four parts of 0.1 M sodium acetate results in a buffer with pH 4.46. The rProtein A column was eluted with a buffer having pH 3.0 (0.1 M acetic acid).

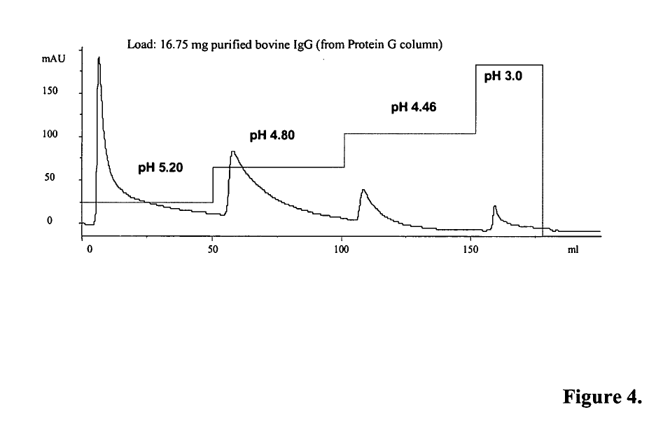

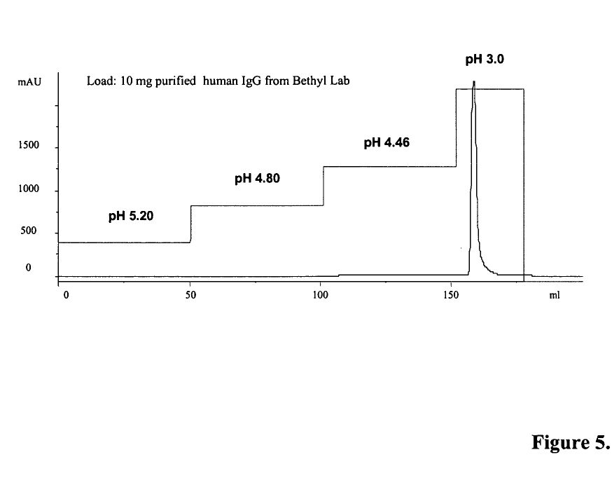

The elution profile of bovine IgG is shown in FIG. 4, and the elution profile of human IgG is shown in FIG. 5. IgG bound very weakly to the rProtein A column while human IgG bound very tightly to the rProtein A column (compare the Y scale in FIG. 4 to Y scale in FIG. 5). The majority of bovine IgG did not bind to rProtein A column during the feedstock application. The bovine IgGs that were bound to rProtein A column were washed off the column with low pH buffers (pH 5.20, 4.80, and 4.46). No significant amount of human IgG was detected in the flow-through of rProtein A column during feedstock application. Measurement of total protein indicated that the percentage of total bovine protein (bovine IgG and other non-specific proteins) in the pH 3.0 elution fraction (FIG. 4) from rProtein A column was only 2-3% of total human IgG eluted by pH 3.0 (FIG. 5) from the rProtein A column.

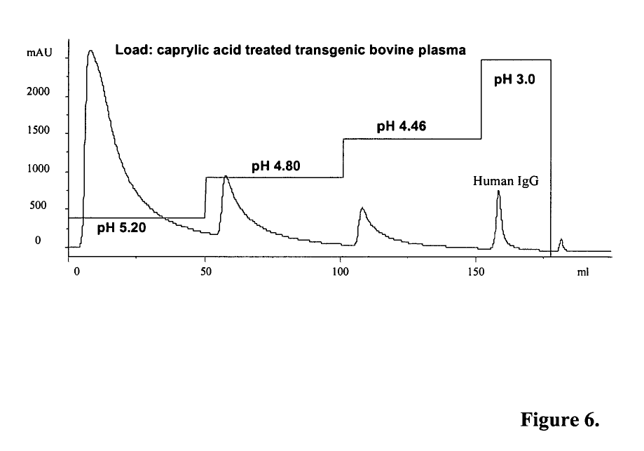

Current transgenic bovine plasma contains 10-30 μg/ml of human IgG and 10-20 mg/ml bovine IgG. Using our novel system for the expression and production of human IgG in transgenic bovine plasma, we precipitated the transgenic bovine plasma with CA (see above), and the supernatant was applied onto a 5 ml rProtein A column, washed, and eluted as described above. Highly purified human IgG was obtained in the pH 3.0 elution fraction (see pH 3.0 peak in FIG. 6). Thus, rProtein A column chromatography in combination with low pH washes and pH 3.0 elution is very effective in separating bovine IgG from human IgG when CA treated transgenic bovine was used as a feedstock.

### Example 4

### Removal of IgG Dimers and Aggregates by MEP HyperCel™ Chromatography

IgG dimers and aggregates are difficult to separate from IgG monomers during standard IgG manufacturing processes. We have discovered that an IgG binding resin, MEP HyperCel™, does not bind or very weakly binds IgG dimers and aggregates under conditions of pH 6 to 8.5. The MEP HyperCel™ resin was obtained from Ciphergen Biosystems, Inc and has 4-Mercapto-Ethyl-Pyridine as an affinity ligand. 4-Mercapto-Ethyl-Pyridine has a hydrophobic tail and an ionizable headgroup which is uncharged and hydrophobic at physiological pH. Under acidic pH conditions, the ligand takes on a positive charge, as does the IgG, and electrostatic repulsion occurs, causing the dissociation of the IgG. Commercially available human IVIG contains some IgG dimers and was used as IgG dimer feedstock. IgG aggregates were generated by incubating IgG feedstocks at 60° C. for 1 to 3 hours at pH 8 to 8.5. IgG feedstocks tested in this example included the commercially available human IVIG, CA-treated human plasma, and CA-treated bovine plasma. Typically, the IgG feedstock from CA treated plasma was adjusted to pH 8.5 with 1 M Tris and to 0.15 M NaCl with 4 M NaCl, and then incubated in a 60° C. water bath for 2 hours. For IVIG, the feedstock was diluted in 50 mM Tris-HCl, pH 8.5, 0.15 M NaCl, and then incubated in a 60° C. water bath for 2 hours. After cooling down, the heat-treated feedstock was applied onto MEP HyperCel™ column, rProtein A, or Protein G column for IgG purification. In the case of the MEP HyperCel™ column, the IgG feedstock was applied onto the column that had been equilibrated with 50 mM Tris-HCl, pH 8.5, 0.15 M NaCl, followed by washing with (1) 50 mM Tris-HCl, pH 8.5, 0.15 M NaCl, and (2) 50 mM sodium phosphate, pH 6.0. The column was then eluted with 50 mM Na-acetate, pH 4.4 and 50 mM glycine-HCl, pH 3.0, respectively. For the Protein A or Protein G column, the IgG feedstock was applied onto the column that had been equilibrated with PBS, washed with PBS, and eluted with 50 mM glycine-HCl, pH 3.0. All eluted protein peaks were brought to neutral pH by adding 1 M Tris-HCl, pH 8.0 and analyzed on a size exclusion column. Some unbound materials (flow-through) were also analyzed on a size exclusion column.

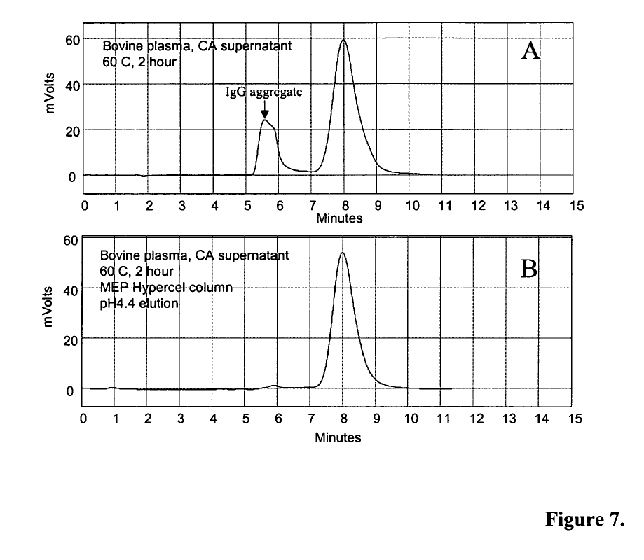

FIG. 7 shows the SEC result of bovine IgG purified from heat-treated (60° C. for 2 hours) CA supernatant by MEP HyperCel™ affinity chromatography. IgG samples were analyzed by size exclusion chromatography on a TSK-GEL G3000SW column connected to and controlled by a Shimadzu VP HPLC system. The protein peak at the retention time of approximately 8 minutes represents IgG monomers, while the protein peak at retention time of approximately 5.5 minutes represents IgG aggregates. Heat-treated IgG feedstocks contained relatively high amounts of IgG aggregates, while IgG aggregates almost disappeared from IgG feedstock purified by MEP HyperCel™ column. These results demonstrate the effectiveness of the MEP Hypercel™ column in removing IgG aggregates from monomeric IgG in a bovine IgG feedstock.

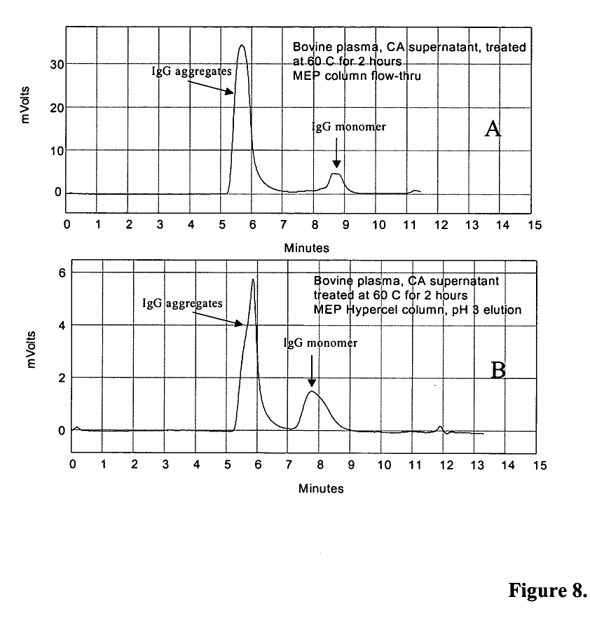

FIG. 8 shows the SEC result of bovine IgG samples in a MEP HyperCel™ column flow-through (unbound material) and pH 3.0 eluted fraction. The IgG feedstock was analyzed by size exclusion chromatography on a TSK-GEL G3000SW column connected to and controlled by a Shimadzu VP HPLC system. The protein peak at the retention time of approximately 8 minutes represents IgG monomers, while the protein peak at retention time of approximately 5.5 minutes represents IgG aggregates. The majority of IgG aggregates were in the flow-through fraction, and a small fraction of IgG aggregates bound to MEP HyperCel column. This fraction was eluted off by 50 mM glycine-HCl, pH 3.0.

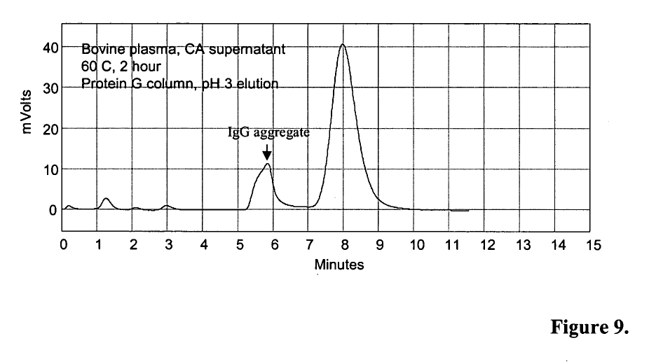

FIG. 9 shows the SEC result of bovine IgG purified from heat-treated (60° C. for 2 hours) CA supernatant by Protein G affinity chromatography. The IgG sample was analyzed by size exclusion chromatography on a TSK-GEL G3000SW column connected to and controlled by a Shimadzu VP HPLC system. The protein peak at the retention time of approximately 8 minutes represents IgG monomers, while the protein peak at retention time of approximately 5.5 minute represents IgG aggregates. These results demonstrate that the Protein G column was not effective in resolving IgG aggregates from monom eric IgG.

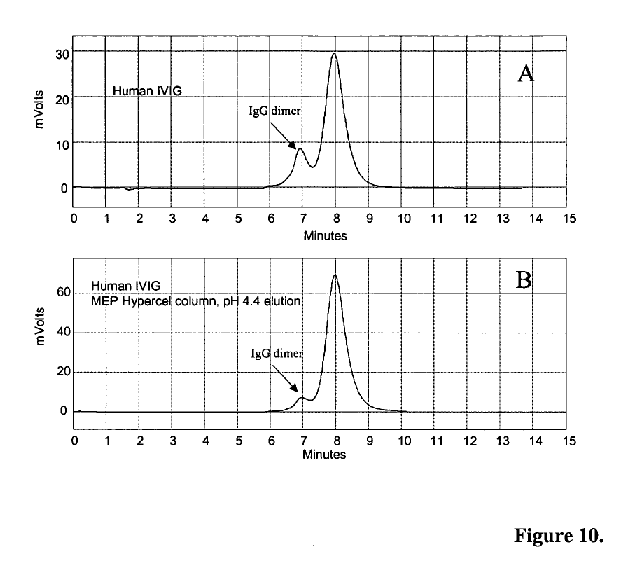

FIG. 10 shows the SEC result of human IVIG purified by MEP HyperCel™ affinity chromatography. The IgG sample was analyzed by size exclusion chromatography on a TSK-GEL G3000SW column connected to and controlled by a Shimadzu VP HPLC system. The protein peak at the retention time of approximately 8 minutes represents IgG monomers, while the protein peak at the retention time of approximately 7 minute represents IgG dimers. Following purification by MEP HyperCel™ column, the relative percent of IgG dimers decreased significantly. Thus, MEP HyperCel™ column is effective for reducing IgG dimers from the human IgG feedstock.

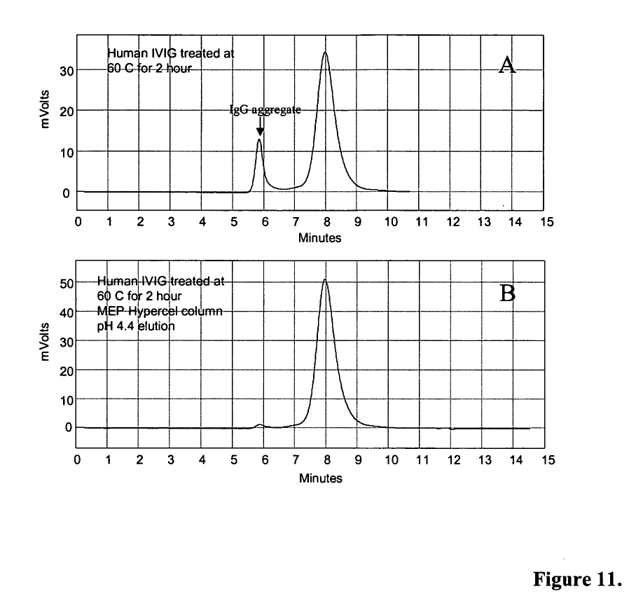

FIG. 11 shows the SEC result of human IVIG purified from heat-treated (60° C. for 2 hours) IVIG feedstock by MEP HyperCel™ affinity chromatography. IgG samples were analyzed by size exclusion chromatography on a TSK-GEL G3000SW column connected to and controlled by a Shimadzu VP HPLC system. The protein peak at the retention time of approximately 8 minutes represents IgG monomers, while the protein peak at retention time of approximately 5.5 minutes represents IgG aggregates. Following purification by the MEP HyperCel™ column, IgG aggregates almost disappeared. Thus, the MEP HyperCel™ column is very effective for removing IgG aggregates from human IgG feedstock.

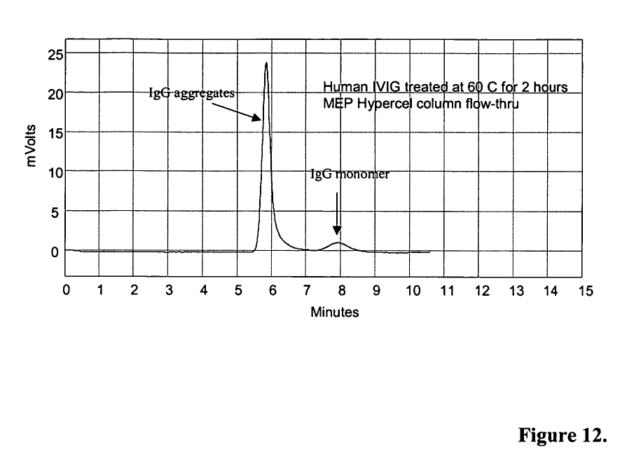

FIG. 12 shows the SEC result of human IgG feedstock from MEP HyperCel™ column flow-through (unbound material). The IgG feedstock was analyzed by size exclusion chromatography on a TSK-GEL G3000SW column connected to and controlled by a Shimadzu VP HPLC system. The protein peak at the retention time of approximately 8 minutes represents IgG monomers, while the protein peak at retention time of approximately 5.5 minutes represents IgG aggregates. All human IgG aggregates were in the flow-through fraction.

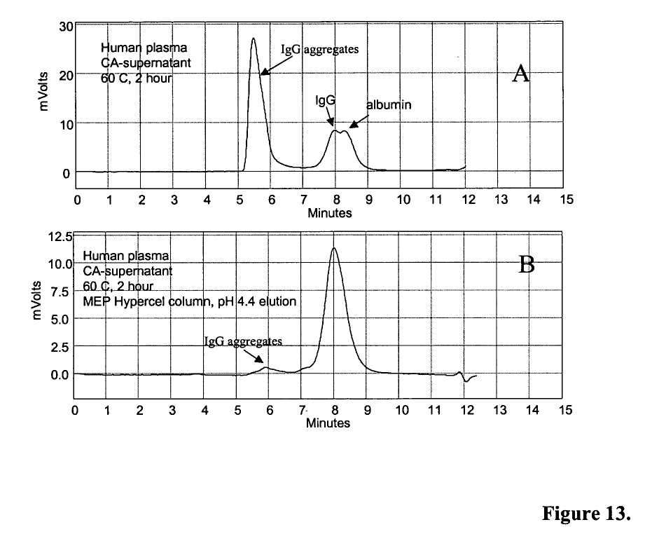

FIG. 13 shows the SEC result of human IgG purified from heat-treated (60° C. for 2 hours) CA supernatant by MEP HyperCel™ affinity chromatography. The IgG samples were analyzed by size exclusion chromatography on a TSK-GEL G3000SW column connected to and controlled by a Shimadzu VP HPLC system. The protein peak at the retention time of approximately 8 minutes represents IgG monomers, while the protein peak at retention time of approximately 5.5 minutes represents IgG aggregates. Heat-treated IgG feedstock contained a lot of IgG aggregates, while IgG aggregates almost disappeared from IgG feedstock after purification by MEP HyperCel™ column, indicating that the MEP HyperCel™ column is very effective for removing IgG aggregates from human IgG feedstocks.

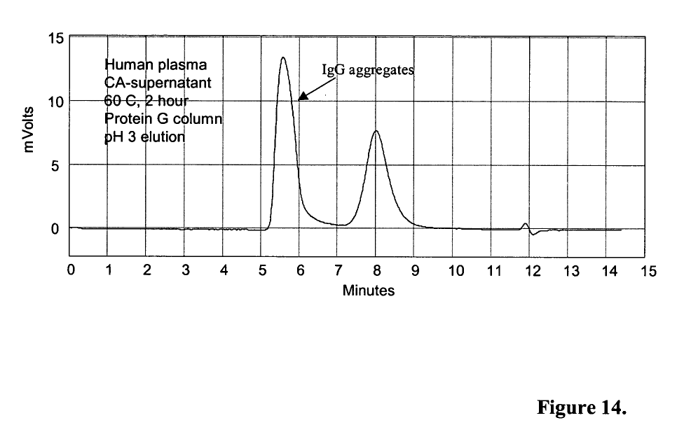

FIG. 14 shows the SEC result of human IgG purified from heat-treated (60° C. for 2 hours) CA supernatant by Protein G affinity chromatography. The IgG sample was analyzed by size exclusion chromatography on a TSK-GEL G3000SW column connected to and controlled by a Shimadzu VP HPLC system. The protein peak at the retention time of approximately 8 minutes represents IgG monomers, while the protein peak at retention time of approximately 5.5 minutes represents IgG aggregates. These results demonstrate that the Protein G column was not effective in removing IgG aggregates from the human IgG feedstock.

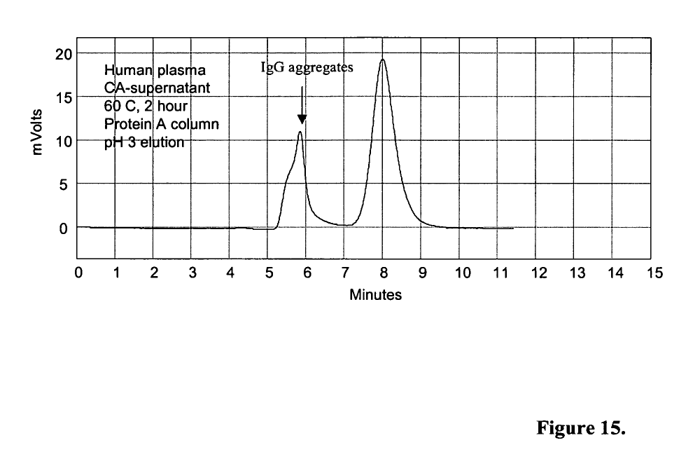

FIG. 15 shows the SEC result of human IgG purified from heat-treated (60° C. for 2 hours) CA supernatant by rProtein A affinity chromatography. IgG sample was analyzed by size exclusion chromatography on a TSK-GEL G3000SW column connected to and controlled by a Shimadzu VP HPLC system. The protein peak at the retention time of approximately 8 minutes represents IgG monomer, while the protein peak at retention time of approximately 5.5 minutes represents IgG aggregates. Thus, the rProtein A column was not effective in removing IgG aggregates from the human IgG feedstock.

### Example 5

### Removal of Bovine IgG and Human/Bovine Chimeric IgG by Anti-Bovine IgG Immunoaffinity Chromatography

Transgenic cattle engineered to produce human IgG express three different types of IgG molecules: bovine IgG (bIgG), human IgG (hIgG), and chimeric IgG (cIgG) that contains either human heavy chain (HC) and bovine light chain (LC) or human LC and bovine HC (FIG. 16). The concentration of hIgG in transgenic bovine plasma ranges from 10 to 30 μg/ml, while bIgG concentration is in the range of 10-20 mg/ml; the concentration of cIgG is unknown.

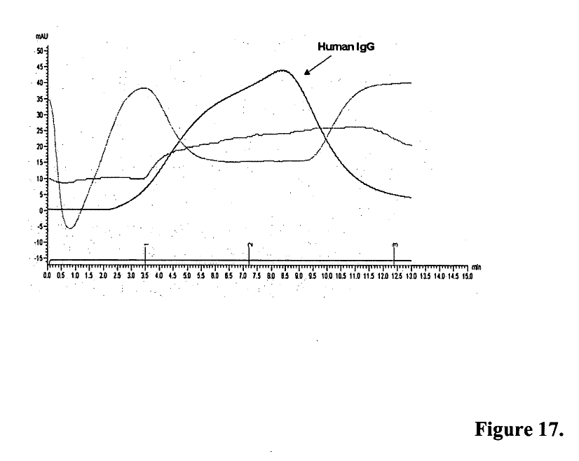

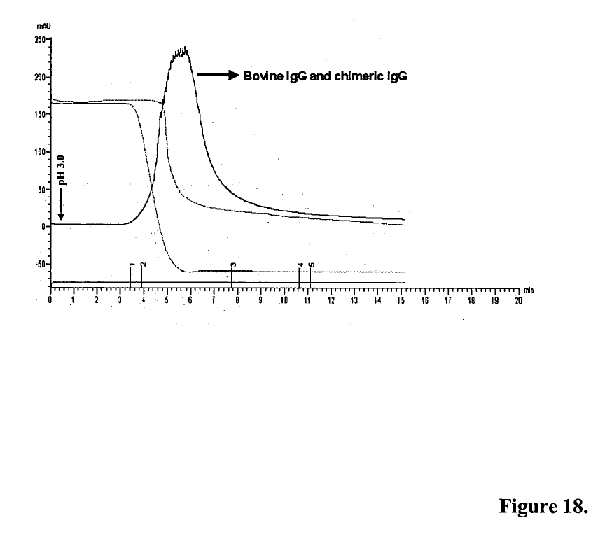

The following methods were developed to purify hIgG substantially free of bIgG and cIgG. Transgenic bovine plasma was diluted and precipitated with 2.5% CA according to a method described by McKinney and Parkinson (supra). The pH of the CA supernatant was adjusted to pH 7.5 to 8.0, and applied onto an rProtein A Sepharose™ column to capture the hIgG and cIgG as in Example 1 and to remove the bIgG and other bovine proteins. The hIgG and cIgG feedstock was eluted from the rProtein A column by pH 3.0 and then adjusted to pH 7.5 to 8.0 and applied onto a horse anti-bIgG Sepharose™ column. The flow-through (unbound material) from the anti-bovine IgG column contained hIgG (FIG. 17), while bIgG and cIgG bound to the column and were eluted off the column with 50 mM Glycine-HCl, pH 3.0 (FIG. 18).

Horse anti-bovine IgG was raised in horses with purified bovine IgG as an antigen. Horse plasma was collected and anti-bovine IgG antibodies were purified by a bovine IgG Affi-Gel immunoaffinity column, followed by affinity strip on a human IgG Agarose column to absorb those antibodies that cross react with hIgG. The affinity purified and stripped anti-bIgG antibody sample, which is only specific for bIgG, was then immobilized onto CNBr-activated Sepharose™ resin to make a horse anti-bovine IgG immunoaffinity column.

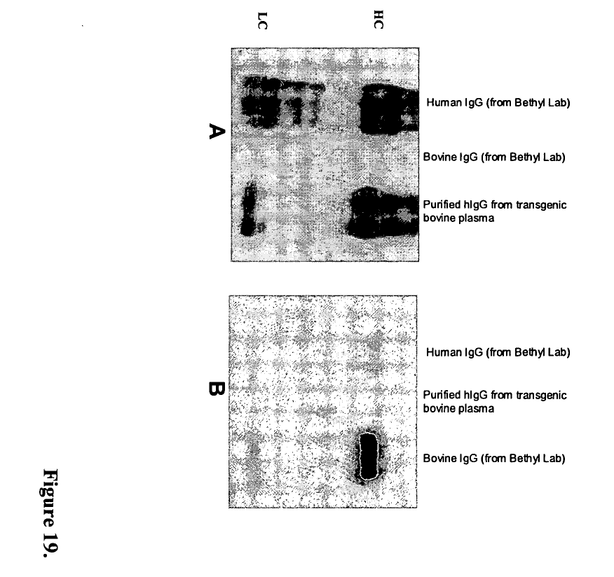

Human IgG purified from transgenic bovine plasma by the above method contains no detectable bovine IgG when analyzed by Western blot (FIG. 19).

### Example 6

### Removal of Bovine IgG and Human/Bovine Chimeric IgG by VHH Ligand Immunoaffinity Chromatography

An alternative approach for separating fully human IgG from non-human (bovine in this example) IgG or non-human/human chimeric IgG was tested using VHH ligands that specifically bind to bovine IgG. VHH ligands are small, single chain proteins having only heavy chain variable regions that behave very similarly to whole antibodies in terms of binding characteristics.

VHH ligands with a high affinity for bovine IgG heavy chain or light chain were produced in collaboration with The Biotechnology Application Centre (BAC) using the methods described in U.S. Patent Application Publication No. 20030078402, and U.S. Pat. Nos. 6,399,763 and 6,670,453 following immunization of llama with bovine IgG. These VHH ligands were purified and immobilized on an affinity chromatography column using a resin, such as NHS Sepharose, and tested for their ability to remove bovine IgG or chimeric IgG from the feedstock, thereby allowing only the human IgG to be collected in the flow-thru.

For these experiments, each VHH ligand was immobilized onto a matrix, and samples (20 ml) containing different amounts of human IgG (hIgG) and bovine IgG (bIgG) were passed through each column and the flow-thru (non-binding fraction) was collected and measured for bIgG by ELISA. Table 7 shows the bovine IgG concentration in parts per million (ppm) calculated based on human IgG in the flow-thru before and after chromatography.

TABLE 7
Removal of bovine IgG from human IgG samples by anti-bovine IgG
light chain VHH column.

| | Pre-VHH, | Post VHH, |
|---|---|---|
| Column/sample | Bovine IgG, ppm | Bovine IgG, ppm |
| LC01 | | |
| 100 mg hIgG + 10 mg bIgG | 100,000 | 90.9 |
| 100 mg hIgG + 5 mg bIgG | 50,000 | 117.3 |
| 100 mg hIgG + 2.5 mg bIgG | 25,000 | 69.9 |
| 100 mg hIgG + 1 mg bIgG | 10,000 | 23.5 |
| 100 mg hIgG + 0.5 mg bIgG | 5,000 | 16.1 |
| 100 mg hIgG + 0.1 mg bIgG | 1,000 | 9.2 |
| LC04 | | |
| 100 mg hIgG + 10 mg bIgG | 100,000 | 87.1 |
| 100 mg hIgG + 5 mg bIgG | 50,000 | 94.1 |
| 100 mg hIgG + 2.5 mg bIgG | 25,000 | 50.6 |
| 100 mg hIgG + 1 mg bIgG | 10,000 | 30.8 |
| 100 mg hIgG + 0.5 mg bIgG | 5,000 | 26.1 |
| 100 mg hIgG + 0.1 mg bIgG | 1,000 | 17.2 |

### Example 7

### Further Purification of IgG Using Membrane-Based Electrophoresis

We have also discovered that membrane-based electrophoresis, such as the Gradiflow system from Life Therapeutics Inc., can be used to purify IgG from transgenic bovine plasma after precipitation with CA to remove the majority of bovine plasma proteins including BSA. Transgenic bovine plasma was treated with CA, followed by pH adjustment to 7.5-8.0, and incubation at 60° C. for 1 to 2 hours to generate IgG aggregates. BSA was also added to the heat-treated sample. The sample that contains IgG aggregates and BSA was buffer exchanged into 41 mM MOPSO, 14 mM β-Alanine, pH 5.0 and purification was achieved by running a bench scale Gradiflow GF 400 system or a pilot scale GF100 system. The purified sample was analyzed by HPLC size exclusion chromatography, BSA ELISA, and HCP ELISA.

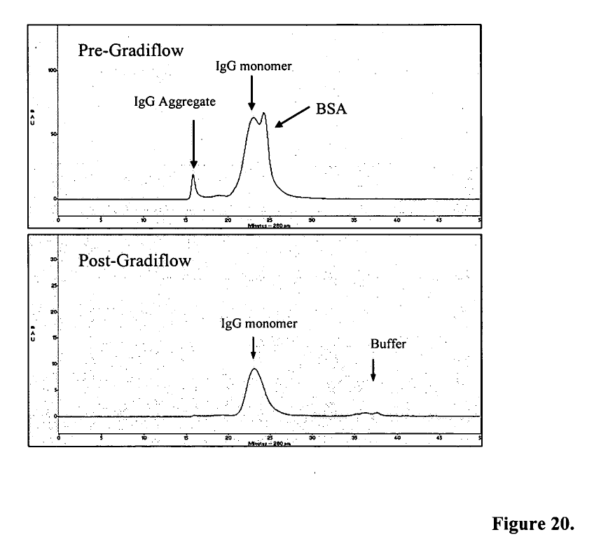

The results presented in FIG. 20 and Table 8 demonstrate that IgG aggregates, BSA, and host contaminating proteins can be separated from IgG monomers during the IgG purification process using membrane-mediated electrophoresis. Thus, membrane-mediated electrophoresis is useful as an effective purification or “polishing step” step for methods of IgG purification.

TABLE 8
Clearance of BSA and HCP by Gradiflow System.

| Sample | BSA, ppm | HCP, ppm |
|---|---|---|
| Pre-Gradiflow | 32616 | 7050 |
| Post-Gradiflow Run 33a | 161 | 1140 |
| Post-Gradiflow Run 33b | 140 | 1010 |
| Post-Gradiflow Run 33c | 134 | 530 |

ppm: part per million, calculated based on amount of total protein.

Membrane-mediated electrophoresis was also shown to effectively remove virus and DNA from the feedstock. Stock solutions of live porcine parvovirus (PPV) and extracted genomic canine parvovirus (CPV) DNA were prepared and were spiked into CA treated starting material in a 1 in 10 ratio (i.e. 10% v/v spike). Table 9 shows the results of PCR analysis and viral infectivity assays conducted for samples pre- and post-Gradiflow system. PCR and infectivity analysis showed that there was no presence of PPV or CPV DNA in any post Gradiflow purified samples. According to endpoint titration of PCR product the log reduction of PPV is estimated to be >5 log, and CPV DNA greater than 5 log, while the log reduction is >3.7 when viral titers were measured by infectivity assay. Thus, these results clearly indicate that the Gradiflow system is also effective in clearing viruses and DNA during the IgG purification process.

TABLE 9
Summary of viral and DNA clearance results*

| | log Reduction by | log Reduction by |
|---|---|---|
| Analysis method | PCR | infectivity |
| PPV virus by PCR | >5 log | NA |
| PPV virus by | NA | >3.74 |
| infectivity | | |
| CPV DNA by PCR | >5 log | NA |

*Results are the average of three runs. NA: not applicable.

### OTHER EMBODIMENTS

From the foregoing description, it will be apparent that variations and modifications may be made to the invention described herein to adopt it to various usages and conditions. Such embodiments are also within the scope of the following claims.

All publications and patents mentioned in this specification are herein incorporated by reference to the same extent as if each individual publication or patent was specifically and individually indicated to be incorporated by reference. Other embodiments are within the claims.

1. A method for purifying immunoglobulin G (IgG) from a feedstock comprising:
(a) adjusting the pH of said feedstock to be within about pH 4.0 to 5.5;
(b) contacting the pH-adjusted feedstock from step (a) with a mono or polyalkanoic acid having from 4 to 12 carbon atoms in an amount and for a time sufficient to form a precipitate and a supernatant, said supernatant comprising IgG;
(c) separating the supernatant of step (b) from said precipitate using centrifugation or filtration;
(d) contacting the supernatant of step (c) with at least one chromatography resin having an affinity for IgG under conditions, including pH, that allow binding to said chromatography resin of at least some of said IgG in the supernatant solution; and
(e) eluting said IgG from said chromatography resin using an eluent, wherein the eluted solution of step (f) comprises a purified IgG.

2. The method of claim 1, wherein said feedstock is taken from a mammal.

3. The method of claim 2, wherein said mammal is an ungulate.

4. The method of claim 3, wherein said ungulate is a transgenic bovine that produces human IgG.

5. The method of claim 1, wherein said feedstock is selected from the group consisting of plasma, serum, ascites, milk, and cell culture supernatant containing polyclonal or monoclonal antibodies.

6. The method of claim 1, wherein the pH of step (a) is about 4.5 to 4.8.

7. The method of claim 1, wherein said polyalkanoic acid is caprylic acid (CA).

8. The method of claim 7, wherein said CA represent 3 to 10%, calculated as volume of CA solution/volume of the total feedstock solution.

9. The method of claim 1, further comprising adjusting the pH of the supernatant of step (c) to a pH suitable for the chromatography resin of step (d).

10. The method of claim 1, wherein said chromatography resin comprises a ligand selected from the group consisting of Protein A, Protein G, 4-Mercapto-Ethyl-Pyridine, an anti-human IgG antibody, and Protein L.

11. The method of claim 10, wherein said anti-human IgG antibody is a horse anti-human IgG antibody or a llama anti-human IgG antibody.

12. The method of claim 1, wherein said purified IgG from step (e) is at least 80% pure.

13. The method of claim 1, wherein said purified IgG from step (e) contains less than 100 parts per million (ppm) of serum albumin or less than 500 ppm of host contaminating proteins.

14. The method of claim 1, further comprising determining the total protein concentration of said feedstock prior to said step (a), and wherein said pH-adjusted feedstock of step (b) is contacted with said mono or polyalkanoic acid in an amount such that the ratio of mono or polyalkanoic acid to said total protein concentration is about 0.75 to about 2.25.

15. The method of claim 14, wherein said ratio of mono or polyalkanoic acid to said total protein concentration is 1 to 2.25.

16. A method for purifying immunoglobulin G (IgG) from a feedstock comprising:
(a) adjusting the pH of said feedstock to be within about pH 4.0 to 5.5;
(b) contacting the pH-adjusted feedstock from step (a) with a mono or polyalkanoic acid having from 4 to 12 carbon atoms in an amount and for a time sufficient to form a precipitate and a supernatant, said supernatant comprising IgG;
(c) separating the supernatant of step (b) from said precipitate by centrifugation or filtration;
(d) adjusting the pH of the supernatant of step (c) to be within a neutral pH range;
(e) dialyzing the supernatant of step (d) against a buffer having a pH of about 4.5 to about 6.0;
(f) purifying said IgG from the supernatant of step (e) using membrane-mediated electrophoresis; and
(g) collecting said purified IgG.

17. The method of claim 16, wherein said feedstock is taken from a mammal.

18. The method of claim 17, wherein said mammal is an ungulate.

19. The method of claim 18, wherein said ungulate is a transgenic bovine that produces human IgG.

20. The method of claim 16, wherein said feedstock is selected from the group consisting of plasma, serum, ascites, milk, and cell culture supernatant having polyclonal or monoclonal antibodies.

21. The method of claim 16, wherein the pH of step (a) is about 4.5 to 4.8.

22. The method of claim 16, wherein said polyalkanoic acid is caprylic acid (CA).

23. The method of claim 22, wherein said CA represents 3 to 10%, calculated as volume of CA solution/volume of the total feedstock solution.

24. The method of claim 16, further comprising determining the total protein concentration of said feedstock prior to said step (a), and wherein said pH-adjusted feedstock of step (b) is contacted with said mono or polyalkanoic acid in an amount such that the ratio of mono or polyalkanoic acid to said total protein concentration is about 0.75 to about 2.25.

25. The method of claim 16, wherein said purified IgG from step (g) is at least 80% pure.

26. The method of claim 16, wherein said purified IgG from step (g) comprises less than 5% IgG aggregates, less than 100 ppm bovine serum albumin, less than 500 ppm host contaminating proteins, less than 5 ppm DNA, undetectabl transmissible spongiform encephalopathy (TSE), undetectable viral DNA, or undetectable viral particles.

27. A method for purifying human IgG from a feedstock, wherein said feedstock is obtained from a non-human transgenic animal that expresses human IgG, said method comprising:
(a) contacting said feedstock with at least one chromatography resin comprising Protein A as a ligand under conditions that allow binding of said human IgG to said chromatography resin;
(b) washing said chromatography resin with a series of one or more wash buffers of increasing acidity until said washing causes the dissociation of non-human IgG, but not human IgG, from said chromatography resin; and
(c) eluting said human IgG from said chromatography resin using an eluent having a pH that is more acidic than the most acidic wash buffer of step (b), wherein the eluted solution of step (c) comprises purified human IgG.

28. The method of claim 27, wherein prior to step (a), said feedstock is first purified by the following steps:
(i) adjusting the pH of said feedstock to be within about pH 4.0 to 5.5;
(ii) contacting the pH-adjusted feedstock from step (i) with CA in an amount and for a time sufficient to form a precipitate and a supernatant, said supernatant comprising IgG;
(iii) separating the supernatant of step (ii) from said precipitate using centrifugation or filtration; and
(iv) adjusting the pH of the supernatant of step (iii) to be within a neutral pH range.

29. The method of claim 27, wherein said non-human transgenic animal is an ungulate.

30. The method of claim 27, wherein said feedstock is selected from the group consisting of plasma, serum, ascites, and milk.

31. The method of claim 27, wherein the series of wash buffers of step (b) comprises two buffers, the first buffer having a pH of about 5.0 to 6.0 and the second buffer having a pH that is more acidic than the pH of the first buffer.

32. The method of claim 27, wherein the series of wash buffers of step (b) comprises three buffers, the first buffer having a pH of about 5.0 to 6.0, the second buffer having a pH that is more acidic than the pH of the first buffer, and the last buffer having a pH that is more acidic than the pH of the second buffer.

33. The method of claim 27, wherein the eluent of step (c) has a pH of about 2.5 to 3.5.

34. The method of claim 27, wherein said purified human IgG is at least 80% pure.

35. The method of claim 27, wherein said purified human IgG is at least 80% free of non-human or chimeric IgGs.

36. A method for purifying IgG monomers from a feedstock, wherein said feedstock comprises IgG monomers and further comprises IgG dimers or aggregates or both, said method comprising:
(a) contacting said feedstock with at least one chromatography resin with an affinity for IgG, wherein said chromatography resin comprises a ligand having a mercapto group and an aromatic pyridine ring, under conditions that allow binding of at least some of said IgG monomer to said chromatography resin;
(b) washing said chromatography resin with at least one buffer; and
(c) eluting said IgG monomer from said chromatography resin using an eluent having an acidic pH, wherein the eluate obtained from step (c) comprises purified IgG monomers.

37. The method of claim 36, wherein said feedstock is taken from a mammal.

38. The method of claim 37, wherein said mammal is an ungulate.

39. The method of claim 38, wherein said ungulate is a transgenic bovine that produces human IgGs.

40. The method of claim 36, wherein said feedstock is selected from the group consisting of plasma, serum, ascites, milk, and cell culture supernatant comprising polyclonal or monoclonal antibodies.

41. The method of claim 36, wherein prior to step (a), said feedstock is first purified by the following steps:
(i) adjusting the pH of said feedstock to be within about pH 4.0 to 5.5;
(ii) contacting the pH-adjusted feedstock from step (i) with a mono or polyalkanoic acid having from 4 to 12 carbon atoms in an amount and for a time sufficient to form a precipitate and a supernatant, said supernatant comprising IgG;
(iii) separating the supernatant solution of step (ii) from said precipitate; and
(iv) adjusting the pH of the supernatant of step (iii) to be within a neutral pH range.

42. The method of claim 41, wherein said polyalkanoic acid is CA.

43. The method of claim 36, wherein said IgG dimers or aggregates or both are generated in said feedstock during production and purification processes.

44. The method of claim 36, wherein said chromatography resin comprises a 4-mercapto-ethyl-pyridine ligand.

45. The method of claim 44, wherein said chromatography resin further comprises a cellulose support.

46. The method of claim 36, wherein said buffer has a neutral or acidic pH.

47. The method of claim 36, wherein said IgG monomer obtained from step (c) is at least 80% free of IgG dimers or aggregates or both.

48. The method of claim 36, wherein said IgG monomer obtained from step (c) is at least 80% pure.

49. A method for purifying human IgG from a feedstock, wherein said feedstock comprises human and non-human IgG, and wherein said feedstock is taken from a transgenic non-human host that expresses said human IgG, said method comprising:
(a) contacting said feedstock with at least one chromatography resin having an affinity for said human IgG under conditions that allow binding of the human IgG to said chromatography resin;
(b) washing said chromatography resin of step (a) with at least one buffer, wherein said buffer causes the dissociation of IgG from said non-human host, but not human IgG, from said chromatography resin;
(c) eluting said human IgG from said chromatography resin using an eluent having an acidic pH;
(d) adjusting the pH of the eluate of step (c) to a neutral pH;
(e) contacting the pH-neutral eluate of step (d) with at least one chromatography resin comprising an anti-host IgG ligand under conditions that allow binding of at least some of the non-human IgG to said chromatography resin comprising anti-host IgG; and
(f) collecting the flow-through from step (e), wherein said flow-through comprises purified human IgG.

50. The method of claim 49, wherein said chromatography resin of step (a) comprises a ligand selected from the group consisting of Protein A, Protein G, 4-Mercapto-Ethyl-Pyridine, an anti-human IgG antibody, and Protein L.

51. The method of claim 49, wherein said host is a bovine and said anti-host is a horse.

52. The method of claim 49, wherein said feedstock is selected from the group consisting of plasma, ascites, serum, and milk.

53. The method of claim 49, wherein said anti-host IgG ligand of step (e) is a ligand specific for the host IgG heavy chain or light chain.

54. The method of claim 53, wherein said anti-host IgG ligand is a VHH ligand.

55. The method of claim 49, wherein said chromatography resin of step (e) comprises sepharose or agarose.

56. The method of claim 49, wherein said human IgG is at least 80% pure.

57. The method of claim 49, wherein said human IgG is at least 80% free of non-human or chimeric IgG.

58. A method for purifying human IgG from a feedstock, wherein said feedstock comprises human and non-human IgG, and wherein said feedstock is taken from a transgenic non-human host that expresses said human IgG, said method comprising:
(a) contacting said feedstock with at least one chromatography resin comprising an anti-host IgG as a ligand under conditions that allow binding of the non-human IgG to said chromatography resin comprising anti-host IgG; and
(b) collecting the flow-through from step (a), wherein said flow-through comprises said human IgG;
(c) contacting said flow-through of step (b) with at least one chromatography resin having an affinity for said human IgG under conditions that allow binding of at least some of said human IgG to said chromatography resin;
(d) washing said chromatography resin of step (c) with at least one buffer, wherein said buffer causes the dissociation of IgG from said non-human host, but not human IgG, from said chromatography resin;
(e) eluting said human IgG from said chromatography resin of step (d) using an eluent having an acidic pH; and
(f) adjusting the pH of the eluate of step (e) to a neutral pH, wherein said eluate comprises purified human IgG.

59. The method of claim 58, wherein said anti-host IgG ligand of step (a) is a ligand specific for the host IgG heavy chain or light chain.

60. The method of claim 59, wherein said anti-host IgG ligand is a VHH ligand.

61. The method of claim 58, wherein said chromatography resin of step (a) comprises sepharose or agarose.

62. The method of claim 58, wherein said host is a bovine and said anti-host is a horse.

63. The method of claim 58, wherein said feedstock is selected from the group consisting of plasma, ascites, serum, and milk.

64. The method of claim 58, wherein said chromatography resin of step (c) comprises a ligand selected from the group consisting of Protein A, Protein G, 4-Mercapto-Ethyl-Pyridine, an anti-human IgG antibody, and Protein L.

65. The method of claim 58, wherein said human IgG is at least 80% pure.

66. The method of claim 58, wherein said human IgG is at least 80% free of non-human or chimeric IgG.

67. The method of claim 49 or 58, wherein prior to step (a), said feedstock is first purified by the following steps:
(i) adjusting the pH of said feedstock to be within about pH 4.0 to 5.5;
(ii) contacting the pH-adjusted feedstock from step (i) with a mono or polyalkanoic acid having from 4 to 12 carbon atoms in an amount and for a time sufficient to form a precipitate and a supernatant, said supernatant comprising IgG;
(iii) separating the supernatant solution of step (ii) from said precipitate; and
(iv) adjusting the pH of the supernatant of step (iii) to be within a neutral pH range.

68. A method for purifying human IgG from a feedstock, wherein said feedstock is taken from a transgenic non-human host that expresses said human IgG, said method comprising:
(a) contacting said feedstock with at least one chromatography resin comprising at least one ligand specific for the non-human host IgG heavy chain or light chain under conditions that allow binding of said non-human host IgG heavy chain or light chain to said chromatography resin comprising at least one ligand; and
(b) collecting the flow-through from step (a), wherein said flow-through comprises purified human IgG.

69. The method of claim 68, wherein said feedstock is selected from the group consisting of plasma, serum, ascites, and milk.

70. The method of claim 68, wherein said chromatography resin comprises sepharose or agarose.

71. The method of claim 68, wherein said ligand is a VHH ligand.

72. The method of claim 68, wherein said purified human IgG is at least 80% free of non-human or chimeric IgGs.

73. The method of any one of claims 1, 27, 36, 49, 58, and 68, said method further comprising purifying the purified IgG using membrane-mediated electrophoresis.

74. A preparation of purified human IgG made using a feedstock from a non-human transgenic host wherein said preparation has a ratio of human IgG to non-human host IgG of at least 2:1.

75. The preparation of claim 74, wherein said ratio is at least 10:1.

76. The preparation of claim 74, wherein said ratio is at least 100:1.

77. A preparation of purified human IgG made using a feedstock from a non-human transgenic host wherein said preparation has a ratio of human IgG to non-human host IgG of at least 2:1, and wherein said preparation is made according to the methods of any one of claims 1, 16, 27, 36, 49, 58, and 68, or a combination thereof.

78. A preparation of purified human IgG made using a feedstock from a non-human transgenic host wherein said preparation comprises less than 1% non-human IgG or less than 40% chimeric IgG.

79. A preparation of purified human IgG made using a feedstock from a non-human transgenic host wherein said preparation comprises less than 100 ppm bovine serum albumin or less than 5 ppm DNA.

80. A preparation of purified human IgG made using a feedstock from a non-human transgenic host wherein said preparation comprises less than 500 ppm host contaminating proteins.

81. A preparation of purified human IgG made using a feedstock from a non-human transgenic host wherein said preparation comprises undetectable levels of viral DNA, viral particles, or transmissible spongiform encephalopathy.

82. The preparation of claim 74, 78, 79, 80, or 81 wherein said non-human transgenic host is a mammal.

83. The method of claim 82, wherein said mammal is an ungulate.

84. The method of claim 83, wherein said ungulate is a transgenic bovine that produces human IgG.
